(19) United States
(12) Patent Application Publication
(10) Pub. No.: US 2025/0000435 A1
(43) Pub. Date: Jan. 2, 2025

(54) SYSTEM FOR DETECTING AND/OR ASSESSING A SUBDURAL HEMATOMA

(71) Applicant: YEDA RESEARCH AND DEVELOPMENT CO. LTD., Rehovot (IL)

(72) Inventors: Alon Harmelin, Rishpon (IL); Vyacheslav Kalchenko, Rehovot (IL)

(21) Appl. No.: 18/701,580

(22) PCT Filed: Oct. 19, 2022
(86) PCT No.: PCT/IL2022/051105

§ 371 Date: Apr. 15, 2024

(30) Foreign Application Priority Data
Oct. 19, 2021 (IL) 287388

(51) Int. Cl. A61B 5/00; A61B 5/01; A61B 5/053; A61B 5/291; A61B 5/296

(52) U.S. Cl. CPC A61B 5/4064; A61B 5/0062; A61B 5/0082; A61B 5/01; A61B 5/053; A61B 5/291; A61B 5/296; A61B 5/6814; A61B 5/7271; A61B 5/742; A61B 2560/0462; A61B 2562/0271; A61B 2562/046

(57) ABSTRACT

A system for detecting and/or assessing a subdural hematoma, constituted of: a wearable structure, configured to be worn on a head of a subject; an optical sub-system, mounted on the wearable structure and being configured for emitting light towards a predetermined location in relation to the wearable structure, and sensing the emitted light returning from the predetermined location, wherein the optical sub-system being further configured for generating a respective set of signals responsively to interactions of the emitted light with the skull-contained matter of the subject; a radio transceiver sub-system configured for emitting sub-optical radiation and generating a signal responsively to an interaction of the radiation with the skull-contained matter of the subject; and a data processor configured to: analyze the signals of the optical sub-system and the radio transceiver sub-system, and detect and/or assess a subdural hematoma of the subject based on the analysis.

### FIELD AND BACKGROUND OF THE INVENTION

The present invention, in some examples thereof, relates to a medical analysis and, more particularly, but not exclusively, to a system for detecting and/or assessing a subdural hematoma.

A stroke is the rapid loss of brain function due to a disturbance in the blood supply to the brain of subject. It can be due to ischemia (lack of blood flow) caused by a blockage, or a hemorrhage (bleeding within the skull). Ischemic strokes produce cerebral infarctions, in which a region of the brain dies due to local lack of oxygen. Hemorrhagic stroke is caused following blood vessel rupture within the brain.

Hemorrhages on the surface of the brain may cause a condition known as a subdural hematoma (SDH). The subdural space of the human head is the space located between the brain and the lining of the brain. which is referred to as the dura mater (hereinafter referred to as the “dura”). Subdural hemorrhages may have a number of causes. For example, elderly persons may be more susceptible to subdural hemorrhages because as the brain ages it tends to become atrophic and the subdural space between the brain and the dura gradually enlarges. Bridging veins between the brain and the dura frequently stretch and rupture as a consequence of relatively minor head injuries, thus giving rise to a collection of blood in the subdural space. Further, severe linear acceleration or deceleration of the brain can result in the brain moving excessively with respect to the dura, often causing rupture of the bridging veins or the blood vessels on the surface of the brain, which can in turn cause subdural hemorrhages in young, and otherwise healthy individuals.

Subdural blood collections are oftentimes classified as acute subdural hematomas, subacute subdural hematomas, and chronic subdural hematomas. Acute subdural hematomas, which are associated with major cerebral trauma, generally consist primarily of fresh blood. Subacute subdural hematomas are generally associated with less severe injuries than those underlying the acute subdural hematomas. Chronic subdural hematomas (CSDHs) are generally associated with even less severe, or relatively minor, injuries. CSDH usually begins forming several days or weeks after bleeding initially starts. CSDH tends to be less dense liquid consisting of very diluted blood, and doesn't always produce symptoms. Another condition involving a subdural collection of fluid is a hygroma, which is a collection of cerebrospinal fluid (sometimes mixed with blood) beneath the dura, which may be encapsulated.

Currently, stroke and SDH or CSDH are diagnosed by contrast-enhanced computed tomography (CT) scan or by magnetic resonance imaging (MRI).

Over the past three decades, use of microwave imaging for biomedical application imaging modality has been introduced, and enabled development of microwave imaging systems that are capable of generating microwave images of human subjects [DOI: 10.1038/srep2045, DOI: 10.2528/PIERB12022006, DOI: 10.1109/TBME.2018.2809541] Heretofore, microwave imaging has been primarily proposed for distinguishing between cancerous and healthy tissues, typically in the breast.

Another known imaging modality is Transcranial Optical Vascular Imaging (TOVI) [Kalchenko et al., Scientific Reports, 2014;4:5839]. This technique combines laser speckle and fluorescent imaging with dynamic color mapping and image fusion, and was shown to be useful for the visualization of hemodynamic changes, particularly perturbations in cerebral blood flow in mouse brains.

### SUMMARY OF THE INVENTION

In some examples, a system for detecting and/or assessing a subdural hematoma is provided.

In some examples, the system comprises a wearable structure, configured to be worn on a head of a subject.

In some examples, the system comprises an optical sub-system, mounted on the wearable structure and being configured for emitting light towards a predetermined location in relation to the wearable structure.

In some examples, the predetermined location coincides with a predetermined area of the head of the subject.

In some examples, the optical sub-system is configured for sensing the emitted light returning from the predetermined location.

In some examples, the optical sub-system is further configured for generating a respective set of signals responsively to interactions of the emitted light with the skull-contained matter of the subject.

In some examples, the system comprises a radio transceiver sub-system configured for emitting sub-optical radiation.

In some examples, the radio transceiver sub-system is configured for generating a signal responsively to an interaction of the radiation with the skull-contained matter of the subject.

In some examples, the system comprises a data processor configured to analyze the signals of the optical sub-system and the radio transceiver sub-system.

In some examples, the data processor is configured to detect and/or assess a subdural hematoma of the subject based on the analysis.

In some examples, while the optical sub-system is mounted on the wearable structure, the predetermined location is adjustable.

Unless otherwise defined, all technical and/or scientific terms used herein have the same meaning as commonly understood by one of ordinary skill in the art to which the invention pertains. Although methods and materials similar or equivalent to those described herein can be used in the practice or testing of examples of the invention, exemplary methods and/or materials are described below. In case of conflict, the patent specification, including definitions, will control. In addition, the materials, methods, and examples are illustrative only and are not intended to be necessarily limiting.

Implementation of the method and/or system of examples of the invention can involve performing or completing selected tasks manually, automatically, or a combination thereof. Moreover, according to actual instrumentation and equipment of examples of the method and/or system of the invention, several selected tasks could be implemented by hardware, by software or by firmware or by a combination thereof using an operating system.

For example, hardware for performing selected tasks according to examples of the invention could be implemented as a chip or a circuit. As software, selected tasks according to examples of the invention could be implemented as a plurality of software instructions being executed by a computer using any suitable operating system. In an exemplary example of the invention, one or more tasks according to exemplary examples of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF SEVERAL VIEWS OF THE DRAWINGS

Some examples of the invention are herein described, by way of example only, with reference to the accompanying drawings. With specific reference now to the drawings in detail, it is stressed that the particulars shown are by way of example and for purposes of illustrative discussion of examples of the invention. In this regard, the description taken with the drawings makes apparent to those skilled in the art how examples of the invention may be practiced.

In the drawings:

FIG. 1 is a block diagram of a system for radio-optical analysis of an object, according to some examples of the present disclosure;

FIG. 2 is a schematic illustration showing the operation principle of the combination of an optical sub-system and a radio transceiver sub-system, according to some examples of the present disclosure;

FIG. 3 is a schematic block diagram illustrated data flow within a data processor, according to some examples of the present disclosure;

FIG. 4 shows fluence for a 25 cm hematoma obtained by computer simulations performed according to some examples of the present disclosure

FIG. 5 is a schematic illustration of a geometry used during computer simulations performed according to some examples of the present disclosure;

FIGS. 6A-D shows radiation intensity obtained by computer simulations performed according to some examples of the present disclosure;

FIGS. 7A-C show logarithmic based intensity map illustration of optical photons propagation (FIG. 7A) and RF waves propagation (FIG. 7B), and ROC curves graphs (FIG. 7C), obtained by computer simulations performed according to some examples of the present disclosure;

FIG. 8 is a schematic illustration of a bi-layered spherical gelatin phantom, prepared for experiments conducted according to some examples of the present disclosure;

FIGS. 9A-C are images of three variants of the prepared bi-layered gelatin spheres prepared, according to some examples of the present disclosure;

FIGS. 10A and 10B are images of an anthropomorphic human head gelatin phantom with a hematoma, prepared for experiments conducted according to some examples of the present disclosure;

FIGS. 11A-E are images of some working examples of various types of antennas, tested experimentally according to some examples of the present disclosure;

FIGS. 12A and 12B are images of a radio-optical sensor prepared according to some examples of the present disclosure;

FIGS. 13A and 13B are images of an experimental setup used an experiment performed according to some examples of the present disclosure;

FIGS. 14A and 14B are graphs showing the S11 parameter of a butterfly antenna, and a pin antenna, as obtained in an experiment performed according to some examples of the present disclosure;

FIG. 15 is an example screen image showing data acquisition of RF data, for 300measurements using a spherical phantom, as obtained in an experiment performed according to some examples of the present disclosure;

FIGS. 16A-D show ROC curves graphs measured according to some examples of the present disclosure for a butterfly antenna with RF of 100 Mhz, and central NIR wavelengths of 765 nm and 830 nm;

FIGS. 17A-D show ROC curves graphs measured according to some examples of the present disclosure for a butterfly antenna with RF of 1500 Mhz, and central NIR wavelengths of 765 nm and 830 nm;

FIGS. 18A-D show ROC curves graphs measured according to some examples of the present disclosure for a pin antenna with RF of 100 Mhz, and central NIR wavelengths of 765 nm and 830 nm;

FIGS. 19A-D show ROC curves graphs measured according to some examples of the present disclosure for a pin antenna with RF of 1500 Mhz, and central NIR wavelengths of 765 nm and 830 nm;

FIGS. 20A and 20B show ROC curves graphs measured according to some examples of the present disclosure for a brain phantom with RF of 100 Mhz (FIG. 20A) and 1500 Mhz (FIG. 20B);

FIG. 21 illustrates several configuration of sub-optical antennas and optical sources and/or detectors contemplated according to some examples of the present disclosure;

FIGS. 22A and 22B are schematic illustrations of a radio-optical module, according to some examples of the present disclosure.

FIGS. 23A-D illustrate a planar view (FIGS. 23A and 23C) and an isometric view (FIGS. 23B and 23D) of a front side (FIGS. 23C-D) and a back side (FIGS. 23A-B) of a first carrier substrate of a prototype radio-optical module, according to some examples of the present disclosure;

FIGS. 24A and 24B illustrate a back side and a front side of a second carrier substrate of the prototype radio-optical module, according to some examples of the present disclosure.

FIGS. 25A-E illustrate the prototype radio-optical module once assembled according to some examples of the present disclosure.

FIG. 26A-D illustrate a perspective view (FIG. 26A), a side view (FIG. 26B), a top view (FIG. 26C) and a bottom view (FIG. 26D) of a movable platform and a static structure mounted on wearable structure according to some examples of the present disclosure.

FIG. 27 illustrates a representative example of a graphical user interface (GUI) according to some examples of the present disclosure.

FIGS. 28A-C illustrate exemplary synchronization protocols according to some examples of the present disclosure.

### DESCRIPTION OF SPECIFIC EXAMPLES OF THE INVENTION

The present invention, in some examples thereof, relates to a medical analysis and, more particularly, but not exclusively, to a system for radio-optical analysis.

Before explaining at least one example of the invention in detail, it is to be understood that the invention is not necessarily limited in its application to the details of construction and the arrangement of the components and/or methods set forth in the following description and/or illustrated in the drawings and/or the Examples. The invention is capable of other examples or of being practiced or carried out in various ways.

The inventors of this disclosure found that the use of modalities such as MRI, CT and PET for diagnosing stroke and hemorrhages such as SDH and CSDH are not without certain operative limitations, such as logistical, cost and/or safety issues, which would best be avoided.

The inventors of this disclosure have devised a technique for radio-optical analysis of an object, such as, but not limited to, an organ of a mammalian subject. In some examples of the present disclosure the technique can be used for determining hemodynamic characteristics in the organ. For example, when the organ is the brain of the subject, the technique can be used to classify a brain event, e.g., to distinguish between a stroke and a SDH, or between a stroke and CSDH, or between SDH and CSDH. Unlike MRI, CT and PET, the technique devised by the inventors can, in some examples of the present disclosure, be utilized using a wearable structure. For example, when the object is a brain of a mammalian subject, the wearable structure can be a cap wearable on the head of the subject.

At least part of the operations described herein can be can be implemented by a data processing system, e.g., a dedicated circuitry or a general purpose computer, configured for receiving data and executing the operations described below. At least part of the operations can be implemented by a cloud-computing facility at a remote location.

Computer programs implementing the method of the present examples can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pulls these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present examples can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

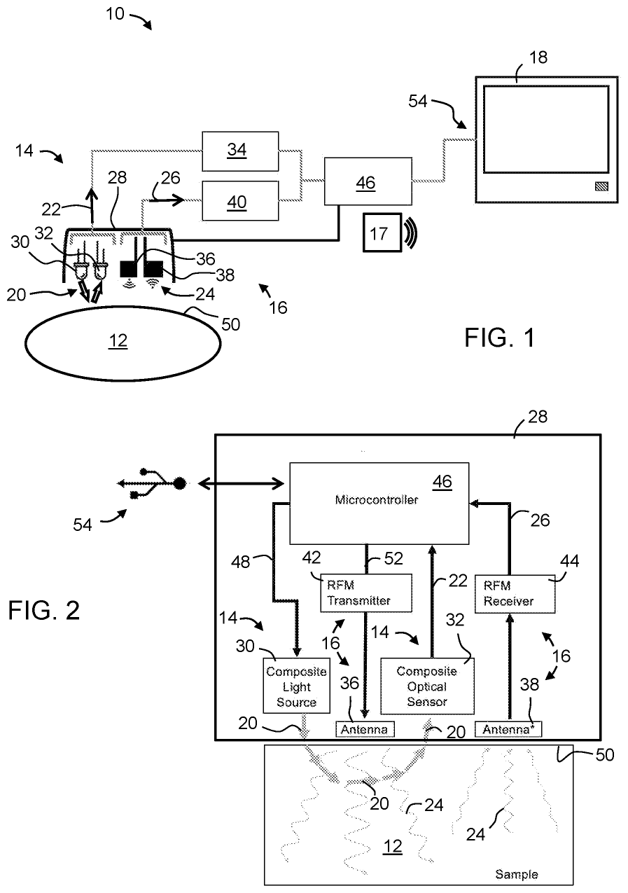

FIG. 1 is a block diagram of a system 10 for radio-optical analysis of an object 12, according to some examples of the present disclosure. Object 12 is typically a brain of a subject, e.g., a mammalian subject, e.g., a human subject.

System 10 typically comprises an optical sub-system 14, a radio transceiver sub-system 16, and a data processor 18. Optical sub-system 14 emits optical radiation (light) 20 to interact with object 12, and generates a signal 22 responsively to the interaction of light 20 with object 12. Radio transceiver sub-system 16 emits sub-optical electromagnetic radiation 24 to interact with object 12, and generates a signal 26 responsively to the interaction of radiation 24 with object 12. Preferably, optical sub-system 14 and radio transceiver sub-system 16 are mounted on a wearable structure 28. When system 10 serves for analyzing the skull-contained matter of the subject, structure 28 is typically configured to be worn on the head of the subject.

The term “skull-contained matter”, as used herein, means any matter that is contained within the skull of the patient. This can include brain tissue, the meninges, blood vessels and/or other present matter.

Optical sub-system 14 and radio transceiver sub-system 16 can operate intermittently, or sequentially. Preferably the operations of optical sub-system 14 and radio transceiver sub-system 16 are synchronized. Representative examples of synchronization protocols suitable for some examples of the invention are described in the Examples section that follows.

Optical sub-system 14 typically comprises a light source system 30 that emits optical radiation 20 and an optical sensor system 32 that receives optical radiation 20, following the interaction with object 12, and generates signal 22. Optical sub-system 14 can also comprise a control circuit 34 that controls the operation of light source system 30, receives signal 22, and transmits it directly or indirectly to data processor 18. In some examples of the present disclosure control circuit 34 performs initial processing to signal 22. For example, control circuit 34 can filter and/or digitize signal 22. Circuit 34 can thus function, at least in part as an analog-to-digital converter. Typically, the digitization employs 12 bits, but higher numbers of bits (e.g., 15, 24, 32, 64) are also contemplated.

Light source system 30 can emit light at one or more wavelengths within the visible and/or near infrared. For example, light source system 30 can emit light at a wavelength range within the range of from about 400 nm to about 1400 nm or from about 635 nm to about 1400 nm. Representative examples of wavelengths suitable for the present examples including, without limitation, 760 nm and 850 nm. Light source system 30 can comprise a multiplicity of light emitting elements. The light emitting elements can emit light at the same or different wavelength bands. Representative examples of light emitting elements including, without limitation, a light emitting diode (LED) packaged or un-packaged die, a laser diode (LD), a vertical-cavity surface-emitting laser (VCSEL) packaged or un-packaged die, an organic LED (OLED) packaged or un-packaged die, a quantum dot (QD) lamp, and the like.

Optical sensor system 32 can comprise a multiplicity of optical sensing elements. Optical sensor system 32 can comprise a multiplicity of optical sensing elements capable of sensing light within any of the aforementioned wavelengths. Representative examples of optical sensing elements suitable for the present examples include, without limitation, a photodiode, an avalanche photodiode, a photovoltaic cell, a light dependent resistor (LDR), a photomultiplier, and the like. Preferably, the optical sensing elements of system 32 are mounted arranged such that two or more different sensing elements are at different distances from light source system 32. The advantage of this example is that it improves the dynamic range, the spatial resolution, and/or the penetration depth

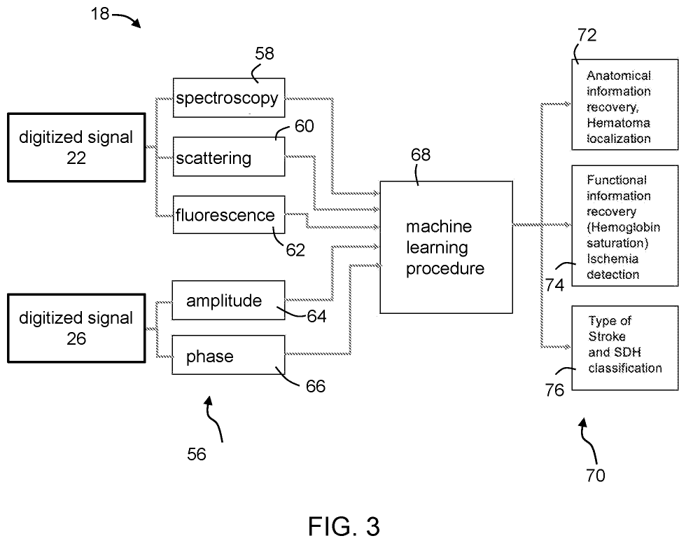

Radio transceiver sub-system 16 preferably comprises one or more antennas 36, 38 that transmit (36) and receive (38) sub-optical electromagnetic radiation 24. Antennas 36, 38 can be printed on a circuit board to improve their radiation pattern. In some examples of the present disclosure system 10 comprises a plurality of antennas, each having different frequency and phase characteristics. Radio transceiver sub-system 16 can also comprise a control circuit 40 that controls the operation of antennas 36, 38, receives signal 26, and transmits it directly or indirectly to data processor 18. Typically, control circuit 40 comprises a radio-transmitter 42 and a radio-receiver 44 (not shown in FIG. 1, see FIG. 2). In some examples of the present disclosure control circuit 40 performs initial processing to signal 26. For example, control circuit 36 can filter and/or digitize signal 26. Thus, similarly to circuit 34, circuit 36 can function, at least in part as an analog-to-digital converter. Typically, the digitization employs 12 bits, but higher numbers of bits (e.g., 15, 24, 32, 64) are also contemplated.

The sub-optical electromagnetic radiation 24 is characterized by a frequency of from about 1 MHz to about 300 GHz, more preferably from about 1 MHz to about 30 GHz, more preferably from about 1 MHz to about 10 GHz, more preferably from about 10 MHz to about 30 GHz, more preferably from about 10 MHz to about 10 GHZ, more preferably from about 100 MHz to about 6 GHz.

In some examples of the present disclosure, radiation 24 is a microwave radiation (e.g., radiation characterized by a frequency of from about 300 MHz to about 300 GHz), and in some examples of the present disclosure, radiation 24 is a radiofrequency radiation (e.g., radiation characterized by a frequency of from about 1 MHz to about 200 MHz).

In some examples of the present disclosure two or more of antennas 36 are configured for transmitting and receiving sub-optical electromagnetic radiation at different frequency bands. For example, one or more antennas can be configured for transmission and receiving of microwave radiation and one or more other antennas can be configured for transmission and receiving of radiofrequency radiation.

In some examples of the present disclosure at least one of optical sub-system 14 and radio transceiver sub-system 16, more preferably both systems 14 and 16 are movable and are configured to emit the respective radiation 20, 24 while assuming a set of different positions relative to wearable structure 28.

When system 14 comprises a plurality of optical sensing elements, they can be movable either independently from each other, or synchronously with each other. When system 16 comprises a plurality of antennas, they can be movable either independently from each other, or synchronously with each other.

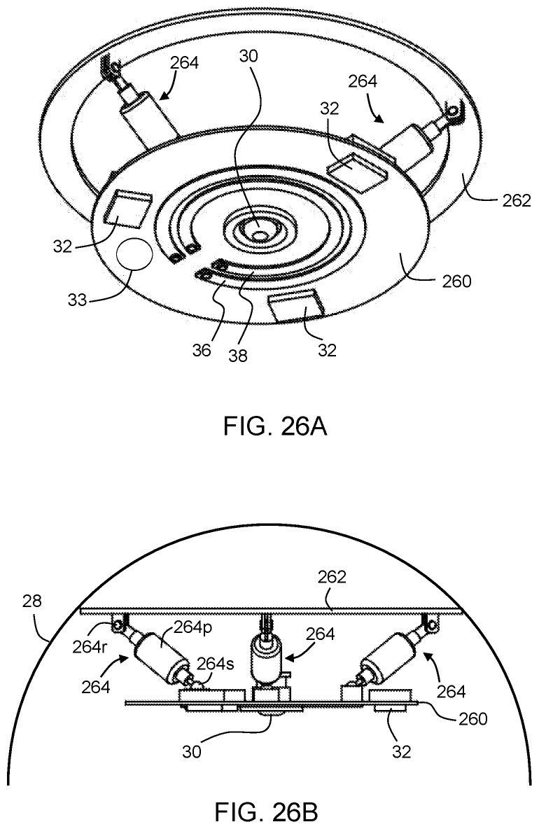

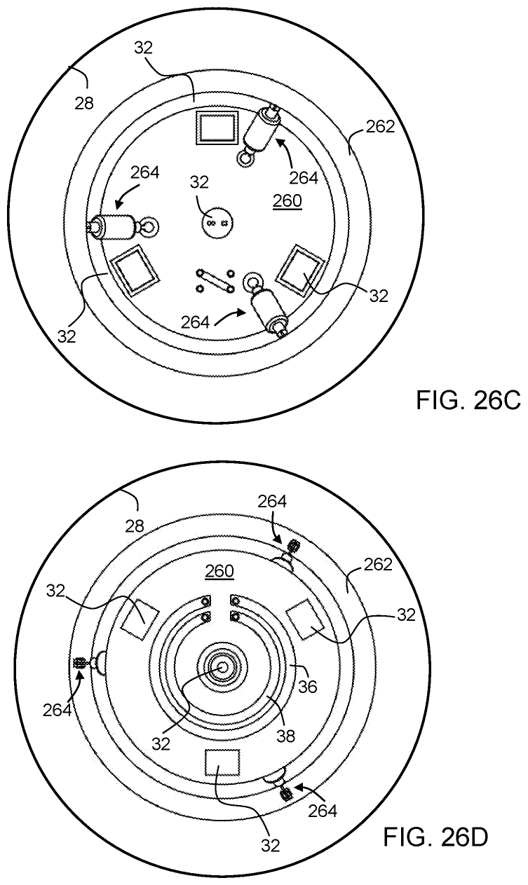

In some examples of the present disclosure wearable structure 28 comprises a platform that movable with respect to a static structure, wherein at least one of optical sub-system 14 and radio transceiver sub-system 16, is mounted on the platform. This example is illustrated in FIGS. 26A-D, showing a perspective view (FIG. 26A), a side view (FIG. 26B), a top view (FIG. 26C) and a bottom view (FIG. 26D) of a movable platform 260 and a static structure 262, mounted on wearable structure 28, according to some examples of the present disclosure. For clarity of presentation, wearable structure 28 is only shown in the side, top, and bottom views.

In some examples, as illustrated in FIG. 26A, one or more additional sensors 33 are provided. In some examples, sensors/s 33 comprise any, or a combination of: electrical impedance sensors, one or more electroencephalogram (EEG) sensors, one or more electromyography (EMG) sensors and one or more temperature sensors.

Static structure 262 is mounted on the internal surface of wearable structure 28, such that once structure 28 is worn on the head of the subject, the movable platform 260 is below static structure 262 contacts, or is in proximity to, the head. The movable platform 260 is connected to static structure 262 by means of one or more actuators 264, such as robotic arms 264. It is convenient to use such robotic arms, but use of other numbers of arms is also contemplated in some examples of the present disclosure.

Although robotic arms are illustrated and described herein, this is not meant to be limiting in any way, and any suitable type of actuator can be used. In some examples, actuators 264 provide tilting about 3 axes, optionally the axes being orthogonal to each other. In some examples, actuators 264 comprises rotation devices configured to rotate about respective rotational axes. In some examples, actuators 264 provide 3 or more degrees of freedom.

In the representative example that is illustrated in FIGS. 26A-D and that is not to be considered as limiting, each arm 264, is of the revolute-prismatic-spherical (RPS) type, including a revolute joint 264r, a prismatic joint 264p, and a spherical joint 264s (see FIG. 26B), but other types of robotic arms can be employed. The revolute 264r and spherical 264s joints are typically passive, and the prismatic joint 264p is actuated by a main controller 46 (not shown, see FIG. 1).

Arms 264 can be configured to provide one, two, three, or more degrees of freedom for movable platform 260. Typically, arms 264 actuate platform 260 at least in two lateral directions parallel to platform 260, but may also be configured to actuate it vertically (perpendicularly to platform 260) and/or to rotate it about one, two, or three rotational axes (e.g., to provide one or more of a yaw, a pitch, and a roll rotations). Arms 264 can be actuated by any technique known in the art such as, but not limited to, electromechanical actuation, resonant ultrasound actuation, and the like.

Referring again to FIG. 1, data processor 18 receives signals 22 and 26, or a combination thereof, and simultaneously analyzes signals 22 and 26 so as to provide functional and/or structural information describing object 12. In some examples of the present disclosure data processor 18 delineates a boundary within the object that at least partially encompasses a region having functional and/or structural properties that are different from regions outside boundary.

The structural information determined by processor 18 can include a map showing a spatial relationship among one or more structural features within object 12, or an image reconstruction of the interior of object 12. For example, when object 12 is a head the structural information can include an image reconstruction of the head or a portion thereof, e.g., an image reconstruction of one or more regions of the subdural space. The functional information provided by processor 18 can include information pertaining to fluid dynamic within object 12. When object 12 is an organ of a mammal the functional information can include hemodynamic characteristics in the organ. When object 12 is the head, data processor 18 can be configured to determine intracranial and/or extracranial physiological and/or pathological conditions related to vascular abnormalities, blood flow disturbances, and/or hemorrhage, such as, but not limited to, subdural and epidural hematomas, and/or stroke. In some examples of the present disclosure processor 18 distinguishes between a stroke and a subdural hematoma within the skull.

In some examples of the present disclosure data processor 18 analyzes signals 22 and 26 separately for each one of the different positions that are assumed by systems 14 and 16. Processor 18 can, for example, determine the functional and/or structural information for each one of these positions, thus providing multiple results, one for each position of systems 14 and 16. Processor 18 can compare the results in order to improve the accuracy. For example, the processor can select, for each region or sub-region within object 12, the result that has the maximal signal-to-noise ratio among the results. Alternatively the processor can improve the accuracy by calculating a weighted average of the results, using a predetermined weight protocol. For example, processor 18 can select the weights for the weighted average based on the signal-to-noise ratio.

Data processor 18 can be local with respect to systems 14 and 16. Alternatively, or additionally, system 10 can include a communication device 17 for transmitting data pertaining to signals 22 and 26 to a remote server (not shown), in which case the simultaneous analysis of the signals is executed by the remote server, and system may be provided without data processor 18. The server can be a central data processor that receives data from multiple systems like system 10 and perform the analysis separately for each system. The results of the analysis (whether executed locally or at the remote server) can be transmitted using communication device 17 to a monitoring location. For example, when the object is a brain of a subject, the results of the analysis can be transmitted to a mobile device held by the subject, to provide the subject with information pertaining to his or her condition. The mobile device can be any of a variety of computing devices (e.g., cell phone, smartphone, handheld computer, laptop computer, notebook computer, tablet device, notebook, media player, Personal Digital Assistant (PDA), camera, video camera, or the like). In various exemplary examples of the invention the mobile device is a smart phone. The results of the analysis can alternatively or additionally be transmitted to a remote location, such as, but not limited to, a computer at a clinic of a physician, or at central monitoring location at a medical facility such as a hospital. The results of the analysis can alternatively or additionally be transmitted to a local monitoring location, such as, but not limited to, the display of processor 18, when processor 18 is positioned, for example, at the bedside of the subject.

In some examples of the present disclosure the information transmitted to the monitoring location includes existence, and optionally and preferably also characteristics (e.g., size and/or location) of at least one of subdural hematoma, epidural hematoma, and stroke, in and around the subject's brain. In some examples of the present disclosure the information transmitted to the monitoring location includes changes in the structure of the subject's skull-contained matter, such as, but not limited to, changes in brain symmetry.

Light source system 30 of optical sub-system 14 can be configured to emit a continuous wave (CW) or pulsed light, as desired. Control signals for operating light source system 30 can be transmitted by control circuit 34. One or more, e.g., all, the individual light emitting elements of source system 30 can be operated simultaneously, thereby providing polychromatic light, or sequentially as desired.

Preferably, optical sub-system 14 is configured for performing at least one of: (i) spectroscopy (either transmitted or diffused), (ii) Static and/or Dynamic Light Scattering (DLS) or laser speckle fluctuations, and (iii) dynamic fluorescence.

Spectroscopy is particularly useful for measurement of existence and optionally levels of one or more materials of interests, such as, but not limited to, oxygen. For example, when object 12 is an organ (e.g., head), spectroscopy can be to measure hemoglobin oxygen saturation, thereby to allow analyzing the metabolism of the organ. Spectroscopy is also useful for detecting ischemic stroke. In these examples, contrast enhanced spectroscopy is optionally and preferably employed. To allow system 14 to perform spectroscopy, source system 30 preferably comprises a plurality of the light emitting elements each emitting non-coherent monochromatic light characterized by a wavelength band. Representative examples of wavelength bands suitable for the present examples including, without limitation, λ+Δλ, where λ can be any subset of 750,800, 830, 850, 900, 950 nm, and Δλ is about 0.12 or 0.052, or any value from about 10 nm to about 20 nm. For example, the light emitting elements in this example can be LEDs.

For dynamic light scattering, the light emitting elements preferable include one or more laser sources. Dynamic light scattering is particularly useful for detecting motion of red blood cells motion inside blood vessels, and can therefore provide a complementary information about blood flow inside the tissue.

Static and/or Dynamic Light Scattering is particularly useful for the detection of fluid dynamic properties, such as, but not limited to, changes in flow and/or perfusion. For example, when object 12 is an organ (e.g., head), Static and/or Dynamic Light Scattering can be used for detecting changes in blood flow and/or changes in blood perfusion. To allow system 14 to perform Static and/or Dynamic Light Scattering, source system 30 preferably comprises one or more light emitting element each emitting a coherent monochromatic light characterized by a wavelength band that is narrower than the characteristic wavelength band of the non-coherent light emitting elements. For example, the light emitting elements in this example can be a LD.

Dynamic fluorescence is also useful for detecting fluid dynamic properties, optionally and preferably, but not necessarily, in addition to the Static and/or Dynamic Light Scattering. When Dynamic fluorescence is employed one or more fluorescent molecules are administered to the object, and light source system 30 is selected or configured to emit light within the absorption spectrum of the fluorescent molecules. For example, one or more of the light emitting elements of source system 30 can be provided with an excitation optical filter selected in accordance with the respective fluorescent molecules. When dynamic fluorescence is employed, one or more of the optical sensing elements of optical sensor system 30 can be provided with an emission optical filter selected in accordance with the respective fluorescent molecules.

The advantage of radio transceiver sub-system 16 is that it transmits radiation that its propagation through material depends on the dielectric properties of the material (e.g., permittivity, conductivity, inductivity). This allows data processor 18 to analyze the radiation and provide functional information describing object 12. The dielectric properties can be determined, for example, by analyzing signal 26 to determine amplitude and/or phase parameters such as, but not limited to, S-parameters (e.g., S11, S12, S22, S21) and the like.

In particular, when the object is an organ (e.g., head), the propagation of radiation 24 through the organ depends on the organ depends on the dielectric properties of the biological material in the organ. Thus, data processor 18 can analyze radiation 24 to identify shifts in one or more dielectric properties (e.g., permittivity, conductivity, inductivity) of the object, and phase of the electromagnetic wave. For example, since the dielectric properties of hematomas and bleeding regions are significantly different from the dielectric properties of the brain matter, the skull and the skin, such identified shifts allow identifying hematomas and bleeding regions and distinguishing between those regions other regions.

Typically, control circuit 40 is configured to irradiate object 12 (via antennas 36) by sub-optical electromagnetic radiation at power that is sufficiently low (e.g., less than 0.1 W more preferably 0.01 W more preferably less than 0.001 W) or so as not to induce thermal effects in object 12. However, in some cases it is desired to induce thermal effects. This is particularly useful for the detection of fluid dynamic properties, e.g., by means of Static and/or Dynamic Light Scattering. Thermal effects are optionally and preferably induced using pulsed sub-optical electromagnetic radiation. In these examples the average power of the sub-optical electromagnetic radiation is from about 3 W to about 6 W or from about 4 W to about 5 W, e.g., about 4.5 W. Thus, in some examples of the present disclosure control circuit 40 is configured to irradiate object 12 via antennas 36 by pulsed sub-optical electromagnetic radiation selected for inducing thermal micro-expansions in object 12.

Both control circuits 34 and 40 are optionally and preferably controlled by main controller 46, preferably a microcontroller, which transmits operation signals to control circuits 34 and 40 in accordance with an irradiation protocol, and receives from circuits 34 and 40 signals indicative of the radio waves and optical waves collected by radio antenna 38 and optical sensor system 30.

FIG. 1 also shows a communication channel between main controller 46 and wearable structure 28, to indicate that controller 46 can also control, as stated, the robotic arms 264 (shown in FIGS. 26A-D) to actuate the movable platform 260 with respect to the static structure 262 that is mounted on wearable structure 28.

The communication between processor 18 and controller 46 is optionally and preferably bilateral, wherein controller 46 can transmit information pertaining to the state of system 10 to processor 18, and processor 18 can transmit operation instructions to wherein controller 46. For example, controller 46 can, in some examples of the present disclosure, be configured for transmitting to data processor 18 information pertaining to the position of systems 14 and 16, or the position of the movable platform 260, and data processor 18 can provides the functional and/or structural information also based on the received positions. For example, processor 18 can divide the volume of the object 12 to a plurality of volume elements, determine for each volume element, the position of systems 14 and 16 that is closest to the volume element among all other positions, and determine the functional and/or structural properties of each volume element based on signals 22 and 24 that are obtained during a time-period at which systems 14 and 16 were closest to the volume element.

In some examples of the present disclosure data processor 18 calculates a signal-to-noise ratio for each of the positions, and instructs controller 46 to control system 14, and/or system 16, and/or movable platform 260, to assume a position based on the calculated signal-to-noise ratio. For example, processor 18 can compare the calculated signal-to-noise ratio to a predetermined threshold, and instruct controller 46 to select a new position of the respective system or platform when the calculated signal-to-noise ratio is less than the threshold

Data processor 18 can also analyze the signals 22 and 26 to determine displacements, and issue an alert, or instruct controller 46 to select a new measurement mode or a position for the respective system or platform, based on calculated motion artifacts. In these examples, processor 18 preferably monitors changes in the respective signals and determines whether or not radio antenna 38 and/or optical sensor system 30 have been displaced, and may also determine the extent of such a displacement. The extent of the displacement can be determined by accessing a library that is stored in a computer readable medium or the memory of processor 38 and that includes a plurality of entries, each comprising a library position and corresponding optical and sub-optical library signal patterns, search the library for library signal patterns that best matches the signal patterns received from systems 30 and 38, and determine the current position of systems 30 and 38 based on the library position of the respective library entry. The determined position can be compared to a previously determined position to determine the extent of the displacement. Such a library can be prepared during a calibration procedure in which signal patterns are characterized and recorded for each of a plurality of different calibration positions.

When the extent of the displacement is above a predetermined threshold, processor 18 can issue an alert signal. A typical situation of a displacement that can trigger an alert is when wearable structure 28 is removed from object 12. When the extent of the displacement is not above the predetermined threshold, processor 18 can instruct the controller 46 to return to the previously determined position or to select a new measurement mode. When processor 18 does not find a matching patterns in the library, processor 18 can instruct the controller 46 to scan the position of systems 30 and 38, until processor 18 finds a match between the signal patterns received from systems 30 and 38 and library signal patterns in the library.

FIG. 2 is a schematic illustration showing the operation principle of the combination of optical sub-system 14 and radio transceiver sub-system 16, according to some examples of the present disclosure. Preferably, optical sub-system 14, radio transceiver sub-system 16, and controller 46 are mounted on wearable structure 28.

Controller 46 transmits, preferably via control circuit 34 (not shown, see FIG. 1), a control signal 48, which is optionally and preferably an electrical signal, to source system 30 to emit light 20. Light 20 interacts (refracted, diffracted, reflected, or scattered) with object 12, and is sensed, following the interaction, by one or more of the optical sensing elements of sensor system 32.

Shown in FIG. 2 is a single optical path between systems 30 and 3, but this need not necessarily be the case, since the interaction of light 20 with object 12 typically results in more than one optical path. For example, each spectral component of light 20 can be redirected differently due to the interaction with object 12, and can also experience more than one type of interaction at one or more points within object 12 (e.g., experience simultaneous refraction and reflection) resulting in ray splitting. Further, since system 30 typically includes, as stated, a multiplicity of light emitting elements, two or more of these light emitting elements can be distributed along the outer surface 50 of object 12, so that light 20 has two or more entry points into object 12, resulting in two or more optical paths for light 20 inside object 12.

Each sensing element of sensor system 32 generates, in response to light 20, signal 22, and these signals are transmitted, optionally and preferably via circuit 34, to controller 46.

Controller 46 also transmits a control signal 52, which is optionally and preferably an electrical signal, to radio-transmitter 42 to emit radiation 24 via one or more of the antennas 36. Radiation 24 interacts (refracted, diffracted, reflected, or scattered) with object 12, and is received, following the interaction, by receiver 44 via one or more of the antennas 38. Since radiation 24 is sub-optical, its penetration depth into object 12 is deeper than light 20. Receiver 44 generates signal 26, in response to the sub-optical radiation picked up by the antennas 38, and transmits these signals controller 46. Controller 46 optionally and preferably digitizes signals 22 and 26 and transmits the digital signals to data processor 18, for example, via s data port 54 of data processor 18.

The acquisition of the digital signals from optical sub-system 14 and radio transceiver sub-system 16 can be simultaneously or sequentially. Multiple units of each of these systems can be placed on the head of the subject, for example, at a distance of from about 2 cm to about 4 cm between adjacent systems of the same type.

The penetration depth of radiation 24 is significantly deeper than light 20. For example, when object 12 is an organ of a mammal (e.g., the head), radiation 24 can penetrate through the object. The electromagnetic waves that form radiation 24 typically undergo multiple reflection and scattering. When object 12 is an organ of a mammal, e.g., the head, in various exemplary examples of the invention signal 26 acquired by system 16 is used by processor 18 for identifying hematomas and bleeding regions through the differences in the dielectric properties between these regions and other regions (e.g., brain matter, the skull and the skin).

Radiation 24 is typically not sensitive to perfusion changes and functional changes in the brain tissue. Such changes are optionally and preferably detected by processor 18 based on signal 22 acquired by optical sub-system 14. Since the penetration depth of light 20 is about 3 cm, signals 22 are typically used by processor 18 for providing information near the surface of object 12. For example, when objects 12 includes the head, signals 22 can be used for determining cortical perfusion changes, and/or distinguishing between SDH, CSDH and Stroke of the middle cerebral artery (MCA).

The attenuation of optical energy is mainly due to the scattering and absorption of near-infrared (NIR) light. One of the contributors of optical contrast during transmitted and diffused spectroscopy is Hemoglobin. In some examples of the present disclosure a NIR fluorophore is introduced to the vasculature near-infrared radiation.

The term “NIR fluorophore” as used herein refers to compounds that fluoresce in the NIR region of the spectrum (e.g., from about 680 nm to 1000 nm).

Representative examples of substances that can be used as NIR fluorophore according to some examples of the present disclosure include, without limitation, indocyanine green (ICG), IRDye™78, IRDye80, IRDye38, IRDye40, IRDye41, IRDye700, IRDye™800CW. Cy5.5, Cy7, Cy7.5, IR-786, DRAQ5NO (an N-oxide modified anthraquinone), quantum dots, and analogs thereof, e.g., hydrophilic analogs, e.g., sulphonated analogs thereof.

The NIR fluorophore enhances the ability of system 10 to detect ischemic stroke based assisted by evaluation of the kinetics in the spectroscopic signal. Specifically, based on the influx and efflux timing of the NIR fluorophore the head's part (e.g., hemisphere) that contains ischemic stroke can be identified.

While NIR fluorophore may be useful, the present inventors found that it is not necessary to use NIR fluorophore in order to determine cortical perfusion changes, and/or distinguish between SDH, CSDH and stroke of the MCA. The inventors found that the use of radio transceiver sub-system 16 allows such a distinction without the use of NIR fluorophore. Thus, according to some examples of the present disclosure at a first stage system 10 is used without introducing a NIR fluorophore, and stroke is identified by simultaneous analysis of both signals 22 and 26. Only in case in which a stroke has been identified, a NIR fluorophore is introduced into the vasculature, and the dynamics of the fluorescent signal acquired by system 14 from the NIR fluorophore is used for the evaluation of more fine parameters of blood flow abnormalities. The advantage of these examples is that in case no stroke is identified, the NIR fluorophore is not introduced into the vasculature.

One of the contributors for the Static and/or Dynamic Light Scattering signal is the level of motion of red blood cells. Preferably, a Static and/or Dynamic Light Scattering signal acquired by optical sub-system 14 is transferred to data processor 18 for information recovery, image reconstruction and analysis.

In some examples of the present disclosure optical sub-system 14 serves as a proximity sensor. In these examples, processor 18 optionally and preferably analyze signal 22 to determine the proximity between wearable structure 28 and the head. When optical sub-system 14 is used as a proximity sensor the wavelength of the optical radiation 20 emitted by system 14 is preferably selected such as to reduce the likelihood for optical radiation 20 to penetrate into object 12.

This can be done, for example, by executing a proximity sensing procedure, in which system 14 is controlled to emit the shortest possible wavelength and with intensity that is less than a predetermined threshold. Alternatively, system 14 can emit a plurality of wavelengths and processor 18 can determine the proximity by analyzing the components of signal 22 that correspond to the shortest wavelengths.

In some examples of the present disclosure both optical sub-system 14 and radio transceiver sub-system 16 serve, collectively as a combined proximity sensor. In these examples, the proximity sensing procedure includes emission of both type of radiations, preferable at the shortest possible wavelengths and with intensity that is less than a predetermined threshold, and processor 18 can determine the proximity by analyzing the respective signals.

Data processor 18 can, in some examples of the present disclosure, analyze signal 26 (received from radio transceiver sub-system 16) to determine whether wearable structure 28 is mounted on a living head. This can be done by determining the dielectric properties of the media though which radiation 24 has been propagating before it was picked up by the antennas 38. When the dielectric properties are characteristic to a brain tissue, processor 18 can determine that structure 28 is mounted on a living head, and when the dielectric properties are not characteristic to a brain tissue, processor 18 can determine that structure 28 is not mounted on a living head. These examples are advantageous because they can reduce the likelihood of false operation of system 10. For example, processor 18 can issue an alarm signal when it determines that structure 28 is not mounted on a living head.

In some examples of the present disclosure data processor 18 determines the type of the tissue based on the determined dielectric properties. For example, processor 18 can access a database having a plurality of entries, each associating a dielectric property or a set of dielectric properties to a tissue type. Based on the tissue type, processor 18 can instruct controller 46 to control the operation of systems 14 and/or 16 according to a predetermined tissue-specific protocol for illuminating the tissue by the respective radiation. The tissue-specific protocol can include emission timing, emission type (e.g., continues, pulsed), emission intensity, and/or radiation wavelength.

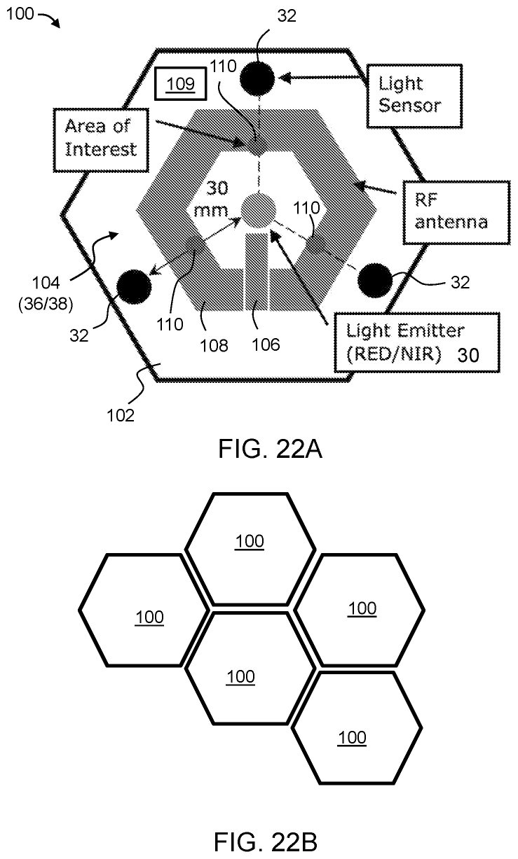

FIGS. 22A and 22B are schematic illustrations of a radio-optical module 100 which incorporates the antenna of radio transceiver sub-system 16, and the light source and optical sensor systems of optical sub-system 14, according to some examples of the present disclosure. Radio-optical module 100 preferably comprises a carrier substrate 102, which is preferably non-conductive. The shape of carrier substrate 102, is optionally and preferably selected to facilitate assembling several modules 100 together, as illustrated in FIG. 22B. The assembled modules can be arranged on wearable structure 28 (not shown). Shown in FIG. 22A are three points 110 marking an area of interest analyzable by module 100. When several modules are assembled (FIG. 22A), the areas of interest of two or more of, more preferably all, the modules combine to define the overall area of interest system 10. The number of modules 100 can be selected based on the size of wearable structure. Typically, but not necessarily, there are from 1 to 20 modules mounted on the wearable structure.

Radio-optical module 100 comprises a conductive pattern 104 formed (e.g., printed, deposited, etc.) on carrier substrate 102. Conductive pattern 104 enacts the antennas 36, 38 of the radio transceiver sub-system, and can be used both for transmitting and receiving the sub-optical electromagnetic radiation. Conductive pattern 104 includes a surrounding portion 108 and a radial portion 106. Preferably, there is a mom-conductive gap between peripheral portion 108 and radial portion 106. Shown in FIG. 22A is a polygonal peripheral portion 108 but round shapes are also contemplated for this peripheral portion. Radial portion 106 typically serves as a feed point for the antenna and peripheral portion 108 typically serves as a collector. Thus radial portion 106 enacts the transmitting antenna 36 and peripheral portion 108 enacts the receiving antenna 38.

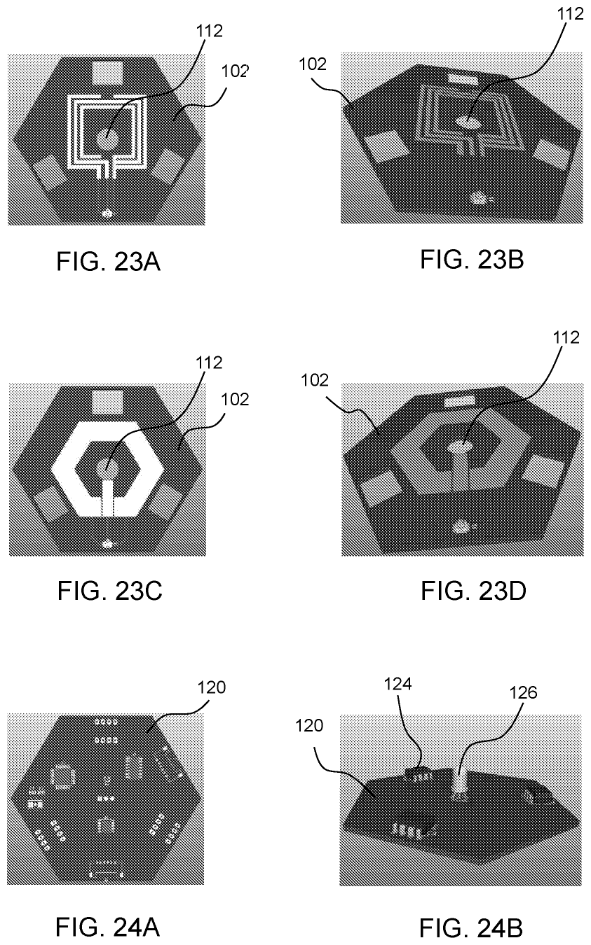

Light source system 30 is positioned at or near to the center of peripheral portion 108. In the schematic illustration of FIG. 22A, which is not to be considered as limiting. light source system 30 is shown as a RED or NIR emitter, but any of the aforementioned types of light source systems, can be employed. Optical sensor system 32 is optionally and preferably distributed peripherally with respect to light source system 30. The distance between light source system 30 and the optical sensing elements of optical sensor system 32 is preferably larger than the distance between light source system 30 and peripheral portion 108, so that optical sensor system 32 is arranged peripherally with respect to pattern 104. Module 100 can also comprises a printed circuit board (not shown, see FIGS. 23A-C), that controls the operation of light source system 30 and arranged the signals received from optical sensor system 32 for transmission. The printed circuit board is typically in addition to control circuit 34, that typically receives signals from all the modules, but the present examples also contemplate configurations in which the printed circuit board of the module transmits the signals directly to processor controller 46, in which case the system may not include control circuit 34.

Typical distance between light source system 30 and the optical sensing elements of optical sensor system 32 is from about 20 mm to about 50 mm, e.g., about 30 mm.

Typical radius of peripheral portion 108 is from about 5 mm to about 20 mm, e.g., about 15 mm.

In some examples of the present disclosure module 100 also comprises a Vector Network Analyzer (VNA) 109. VNA 109 serves for analyzing the signal from the antenna to determine phase shifts or the like. VNA 109 can interact with the antenna either directly or by means of an RF switch (not shown). VNA 109 can generate digital data indicative of its analysis and transmit the data as signal 26, in which case it is not required to digitize signal 26 at circuit 40. Alternatively, circuit 40 can serve as a VNA, in which case it is not required for module 100 to include VNA 109.

FIG. 3 is a schematic block diagram illustrated data flow within processor 18. Signals 22 and 26 are transmitted to data port 54 (not shown, see FIGS. 1 and 2) processor 18 optionally and preferably after they have been digitized by controller 46 (not shown, see FIGS. 1 and 2). Each of these signals is optionally and preferably subjected to several separate feature extraction operations generally shown at 56.

Specifically, signal 22, initially acquired by optical sub-system 14 is subjected to one or more processing operations for extracting features selected from the group consisting of spectroscopic 58, Static and/or Dynamic Light Scattering 60 and fluorescent 62 features. Typically, operations 58, 60 and 62 are synchronized with the operation of controller 46. For example, when optical sub-system 14 is operated in spectroscopic mode (e.g., when source system 30 emits non-coherent monochromatic light), the acquired signal 22 is processed to extract spectroscopic features, when optical sub-system 14 is operated in Static and/or Dynamic Light Scattering mode (e.g., when source system 30 emits a coherent monochromatic light), the acquired signal 22 is processed to extract Static and/or Dynamic Light Scattering features, and when optical sub-system 14 is operated in fluorescence mode (when source system 30 emits light within an absorption spectrum of fluorescent molecules), the acquired signal 22 is processed to extract fluorescent features.

Signal 26, initially acquired by radio transceiver sub-system 16 is subjected to processing operations for extracting is subjected to one or more processing operations for extracting features selected from the group consisting of amplitude 64 and phase 66.

Following the feature extraction operations, the extracted features are optionally and preferably fed to a trained machine learning procedure 68, for simultaneous analysis of all the features.

Representative examples of machine learning procedures suitable for use as machine learning procedure 68 include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, extreme gradient boosting, singular value decomposition methods and principle component analysis. Among neural network models, the self-organizing map and adaptive resonance theory are commonly used unsupervised learning algorithms. The adaptive resonance theory model allows the number of clusters to vary with problem size and lets the user control the degree of similarity between members of the same clusters by means of a user-defined constant called the vigilance parameter.

Following is an overview of some machine learning procedures suitable for the present examples.

Support vector machines are algorithms that are based on statistical learning theory. A support vector machine (SVM) according to some examples of the present disclosure can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present examples includes, without limitation, sequential minimal optimization.

In KNN analysis, the affinity or closeness of objects is determined. The affinity is also known as distance in a feature space between data objects. Based on the determined distances, the data objects are clustered and an outlier is detected. Thus, the KNN analysis is a technique to find distance-based outliers based on the distance of a data object from its kth-nearest neighbors in the feature space. Specifically, each data object is ranked on the basis of its distance to its kth-nearest neighbors. The farthest away data object is declared the outlier. In some cases the farthest data objects are declared outliers. That is, a data object is an outlier with respect to parameters, such as, a k number of neighbors and a specified distance, if no more than k data objects are at the specified distance or less from the data object. The KNN analysis is a classification technique that uses supervised learning. An item is presented and compared to a training set with two or more classes. The item is assigned to the class that is most common amongst its k-nearest neighbors. That is, compute the distance to all the items in the training set to find the k nearest, and extract the majority class from the k and assign to item.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within the datasets. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in providing the functional information describing the object. Symmetrical uncertainty is an algorithm that can be used by a feature selection algorithm, according to some examples of the present disclosure. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present examples aims at selecting the subset of features with the highest impact on functional information describing the object, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present examples contemplate search algorithms that search forward, backward or in both directions. Representative examples of search algorithms suitable for the present examples include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the datasets or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular region is SDH or CSDH or a stroke) or a value. The leaf nodes can also contain additional information about the represented classification such as a confidence score that measures a confidence in the represented classification (i.e., the likelihood of the classification being accurate). For example, the confidence score can be a continuous value ranging from 0 to 1, which a score of 0 indicating a very low confidence (e.g., the indication value of the represented classification is very low) and a score of 1 indicating a very high confidence (e.g., the represented classification is almost certainly accurate).

Regression techniques which may be used in accordance with the present invention include, but are not limited to linear Regression, Multiple Regression, logistic regression, probit regression, ordinal logistic regression ordinal Probit-Regression, Poisson Regression, negative binomial Regression, multinomial logistic Regression (MLR) and truncated regression.

A logistic regression or logit regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regression may also predict the probability of occurrence for each data point. Logistic regressions also include a multinomial variant. The multinomial logistic regression model is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.). For binary-valued variables, a cutoff between the 0 and 1 associations is typically determined using the Yuden Index.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some examples, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future determination of the functional information describing the object. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based techniques generate a new model for each instance, instead of basing predictions on trees or networks generated (once) from a training set.

The term “instance”, in the context of machine learning, refers to an example from a dataset.

Instance-based techniques typically store the entire dataset in memory and build a model from a set of records similar to those being tested. This similarity can be evaluated, for example, through nearest-neighbor or locally weighted methods, e.g., using Euclidian distances. Once a set of records is selected, the final model may be built using several different techniques, such as the naive Bayes.

Neural networks are a class of algorithms based on a concept of inter-connected computer code elements referred to as “artificial neurons” (oftentimes abbreviated as “neurons”). In a typical neural network, neurons contain data values, each of which affects the value of a connected neuron according to connections with pre-defined strengths, and whether the sum of connections to each particular neuron meets a pre-defined threshold. By determining proper connection strengths and threshold values (a process also referred to as training), a neural network can achieve efficient recognition of images and characters. Oftentimes, these neurons are grouped into layers in order to make connections between groups more obvious and to each computation of values. Each layer of the network may have differing numbers of neurons, and these may or may not be related to particular qualities of the input data.

In one implementation, called a fully-connected neural network, each of the neurons in a particular layer is connected to and provides input value to those in the next layer. These input values are then summed and this sum compared to a bias, or threshold. If the value exceeds the threshold for a particular neuron, that neuron then holds a positive value which can be used as input to neurons in the next layer of neurons. This computation continues through the various layers of the neural network, until it reaches a final layer. At this point, the output of the neural network routine can be read from the values in the final layer. Unlike fully-connected neural networks, convolutional neural networks operate by associating an array of values with each neuron, rather than a single value. The transformation of a neuron value for the subsequent layer is generalized from multiplication to convolution.

The machine learning procedure used according to some examples of the present disclosure is a trained machine learning procedure, which receives the features extracted from the digitized version of the signals generated in response to light 20 and radiation 24 and provides output indicative of functional and/or structural information describing the object.

A machine learning procedure can be trained according to some examples of the present disclosure by feeding a machine learning training program with features extracted from digitized version of the signals generated in response to light 20 and radiation 24 following interaction with a cohort of objects (e.g., a cohort of mammalian subjects) for which the functional and structural properties are known. For example, when system 10 is used for analyzing the structure and/or function of a brain, the cohort of objects can be a cohort of objects for which an image reconstruction of the brain is available (e.g., from MRI, CT or PET scans), and for which hemodynamic characteristics within the head, such as, but not limited to, existence or absence of a stroke, a SDH, and/or CSDH, are known (e.g., as determined by analysis of MRI, CT or PET scans). Once the features are fed, the machine learning training program generates a trained machine learning procedure which can then be used without the need to re-train it.

For example, when it is desired to employ deep learning, a machine learning training program adjusts the connection strengths and threshold values among neurons and/or layers of an artificial neural network, so as to produce an output that resembles as much as possible the cohort's known functional and structural properties. When the neural network is a convolutional neural network (CNN), a machine learning training program adjusts convolutional kernels and bias matrices of the CNN so as to produce an output that resembles as much as possible the cohort's known functional and structural properties. The final result of the machine learning training program in these cases is an artificial neural network having an input layer, at least one, more preferably a plurality of, hidden layers, and an output layer, with a learn value assigned to each component (neuron, layer, kernel, etc.) of the network. The trained artificial neural network receives the extracted features at its input layer and provides the functional and/or structural information at its output layer.

Representative types of output that can be provided by the trained machine learning procedure are shown at 70. These include, but are not limited to, anatomical information recovery 72, e.g., location of hematoma etc., functional information recovery 74, e.g., hemoglobin saturation, presence of ischemia etc., and classification 76 of conditions, e.g., stroke, SDH etc.

Also contemplated, are examples in which the trained machine learning procedure provides output pertaining to one or more changes in the brain structure, such as, but not limited to, brain symmetry. For example, depending on the size of the identified hematoma the trained machine learning procedure can determine whether or not a midline shift has occurred, and optionally and preferably also to estimate the such a shift.

In some examples, as described above, optical sub-system is configured for emitting light towards one or more predetermined locations in relation to wearable structure 28. In some examples, the predetermined location coincides with a predetermined area of the head of the subject. Particularly, when wearable structure 28 is secured to the head of the subject, the predetermined location is at a portion of the head of the subject.

In some examples, while optical sub-system 14 is mounted on wearable structure 28, the predetermined location is adjustable. In some examples, as described above, one or more actuators 264 are configured to adjust the predetermined location. In some examples, actuators 264 adjust the position of light source system 30 and/or optical sensing system 32 in relation to wearable structure 28 to thereby adjust the predetermined location.

In some examples, as described above, optical sensing system 32 comprises a plurality of optical sensing elements. In some examples, the one or more actuators 264 adjust the position of the plurality of optical sensing elements independently from each other. The term “independently”, as used herein, means that the position of each element can be adjusted without adjusting the position of another element.

In some examples, as described above, optical sensing system 32 is mounted on a movable platform 260, which is moved by actuators 264. In some examples, actuators 264 can adjust the position of optical sensing system 32 by independently moving the individual optical sensing elements and/or by moving movable platform 260. In some examples, actuators 264 tilt optical sensing system 32 about any of three-axes (optionally 3-axes that are orthogonal to each other).

In some examples, the location where the light is emitted to is adjusted by adjusting the angle of a light source system 30. In some examples, a plurality of light sources are provided (not shown) and the location where the light is emitted to is adjusted by selecting a respective one of the plurality of light sources. In some examples, each of the plurality of light sources is aimed in a respective direction such that the light emitted therefrom covers a predetermined area.

In some examples, the predetermined location which is being analyzed is adjusted by adjusting the position of the optical sensing elements of optical sensing system 32. Thus, regardless of the area covered by the light emitted from light source system 30, optical sensing system 32 detects light from only a predetermined location. In some examples, the location where the light is being sensed from is adjusted by selecting a responsive one of the plurality of optical sensing elements of optical sensing system 32. In some examples, each of the plurality of optical sensing elements is aimed in a respective direction such that light from a predetermined location is sensed.

In some examples, the ability to adjust the location which is being analyzed allows the use of wearable structure 28 without needing a large number optical sub-systems 14.

Similarly, in some examples, a radio transceiver sub-system 16 comprises a plurality of antennas 36 and/or antennas 38.

In some examples, the location where the sub-optical radiation is emitted to is adjusted by adjusting the angle of the respective transmitting antenna 36. In some examples, the location where the sub-optical radiation is to is adjusted by selecting a respective one of the plurality of transmitting antennas 36. In some examples, each of the plurality of antennas 36 is aimed in a respective direction such that the sub-optical radiation emitted therefrom covers a predetermined area.

In some examples, the predetermined location which is being analyzed is adjusted by adjusting the position of the receiving antenna 38. Thus, regardless of the area covered by the sub-optical radiation emitted from antenna 36, antenna 38 detects sub-optical radiation from only a predetermined location. In some examples, the location where the sub-optical radiation is being sensed from is adjusted by selecting a responsive one of the plurality of receiving antennas 38. In some examples, each of the plurality of antennas 38 is aimed in a respective direction such that sub-optical radiation from a predetermined location is sensed.

In some examples, the ability to adjust the location which is being analyzed allows the use of wearable structure 28 without needing a large number optical sub-systems 14 and radio transceiver sub-systems 16.

In some examples, based on the analysis of the signals received from radio transceiver sub-system 16, data processor 18 generates an initial detection notification. Particularly, in some examples, the initial detection notification is an indication that a suspected subdural hematoma is detected. In some examples, responsive to the initial detection notification, controller 46 controls optical sub-system 14 to emit the light to the designated area.

Thus, in such examples, an initial detection is performed by radio transceiver sub-system 16 (with data processor 18) and then further analysis is performed using optical sub-system 14. In some examples, the signals output by radio transceiver sub-system 16 provide superior sensitivity, while the signals output by optical sub-system 14 provide superior specificity. Thus, initial scans can be performed using only radio transceiver sub-system 16, without having to use optical sub-system 14. In some examples, data processor 18 comprises a predetermined model, which receives the signals from both optical sub-system 14 and radio transceiver sub-system 16, and the model analyzes both sets of signals, thereby utilizing the higher sensitivity of optical sub-system 14 and the higher specificity of radio transceiver sub-system 16.

In some examples, based on the analysis of the signals received from optical sub-system 14, data processor 18 generates an initial detection notification. Particularly, in some examples, the initial detection notification is an indication that a suspected subdural hematoma is detected. In some examples, responsive to the initial detection notification, controller 46 controls radio transceiver sub-system 16 to emit the sub-optical radiation. Thus, initial scans can be performed using only optical sub-system 14, without having to use radio transceiver sub-system 16. In some examples this is advantageous since the sub-optical radiation emitted by radio transceiver sub-system 16 may be more harmful than the light emitted by optical sub-system 14.

In some examples, additional information regarding the subject can be provided by sensor/s 33. This information can be derived from ultrasound data, electrical impedance data, EEG data, EMG data, temperature data, or other data.

In some examples, controller 46 controls sensor/s 33 to operate responsive to a respective indication from data processor 18 based on the respective signals of optical sub-system 14 and/or radio transceiver sub-system 16. In some examples, a plurality of sensors 33 are provided and controller 46 individually selects each sensor 33 to operate at a respective predetermined time and/or responsive to a respective indication from data processor 18. As used herein the term “about” refers to ±10%.

The word “exemplary” is used herein to mean “serving as an example, instance or illustration.” Any example described as “exemplary” is not necessarily to be construed as preferred or advantageous over other examples and/or to exclude the incorporation of features from other examples.

The word “optionally” is used herein to mean “is provided in some examples and not provided in other examples.” Any particular example of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various examples of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate examples, may also be provided in combination in a single example. Conversely, various features of the invention, which are, for brevity, described in the context of a single example, may also be provided separately or in any suitable subcombination or as suitable in any other described example of the invention. Certain features described in the context of various examples are not to be considered essential features of those examples, unless the example is inoperative without those elements.

Various examples and aspects of the present disclosure as delineated hereinabove and as claimed in the claims section below find experimental support in the following examples.

### EXAMPLES

Reference is now made to the following examples, which together with the above descriptions illustrate some examples of the invention in a non limiting fashion.

### Computer Simulations

Computer simulations were conducted according to some examples of the present disclosure to investigate propagation of radio waves through the human skull in order to determine the ability of the system of the present examples to detect a subdural hematoma. The following hematoma diameters were simulated: of 6 mm, 10 mm, 25 mm and 35 mm.

The optical sub-system was simulated as providing NIR light at one or more wavelength bands having the following central wavelengths: 750 nm, 850 nm, 950 nm. The width of each wavelength band was not more than 100 nm. The radio transceiver sub-system was simulated as providing radiofrequency radiation at one or more frequency bands having the following central frequencies: 0.5 GHZ, 1 GHZ, 1.5 GHZ, and 2 GHz. The width of each frequency band was less than 1 MHz.

A 100×100×100 mm model was simulated as being filled with different layers (skin, bone, CSF, white/gray matter, hematoma), each layer being characterized by the following set of characteristics: absorption, scattering, anisotropy and refractive index.

The simulation software included MCXLAB [Leiming Yu, Fanny Nina-Paravecino, David Kaeli, Qianqian Fang, “Scalable and massively parallel Monte Carlo photon transport simulations for heterogeneous computing platforms,” J. Biomed. Opt. 23(1), 010504 (2018)]. MCXLAB is the native MEX version of MCX for MATLAB (MCX—Monte Carlo eXtreme—Monte Carlo software for time-resolved photon transport simulations in 3D turbid media powered by GPU-based parallel computing).

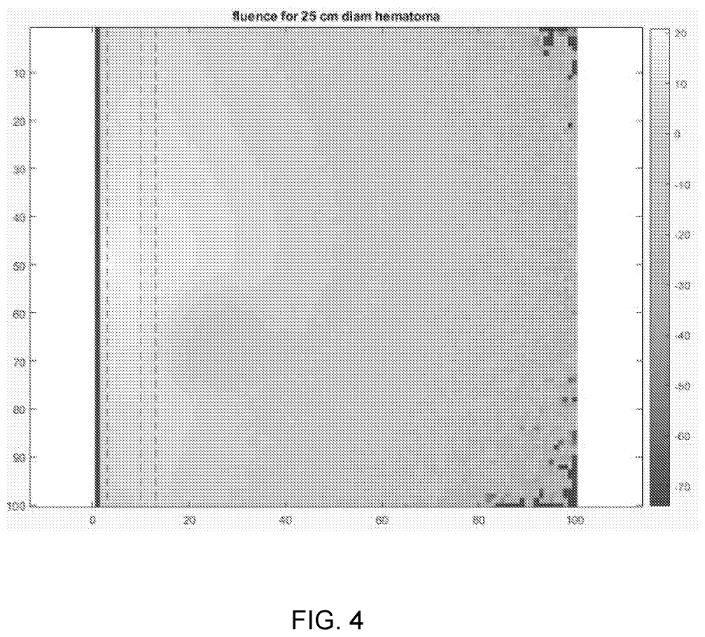

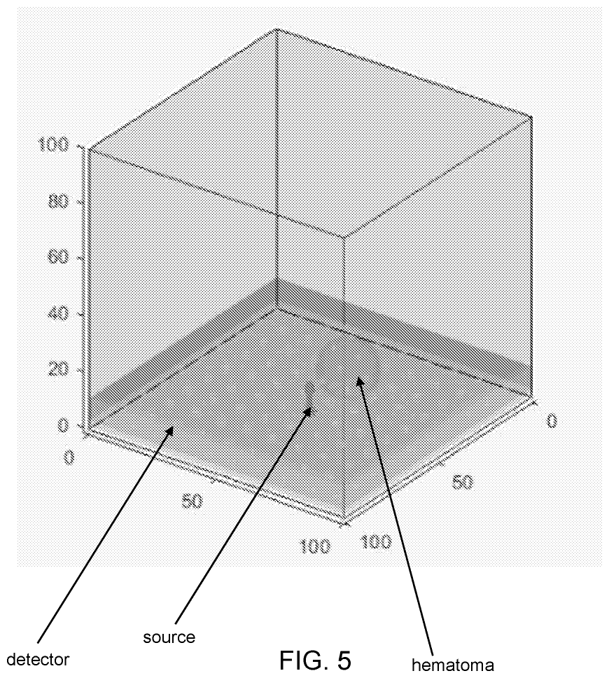

In a first simulation, the model included a scalp (3 mm), a skull (7 mm), CSF (2mm), gray matter (4 mm), and white matter (100 mm), as shown in FIG. 4 The simulation was based on 1 radiation source and 11×11 detectors with 1 cm step, as illustrated in FIG. 5.

Monte Carlo simulation included 6300 different variations of the hematoma size (6, 10, 25 and 35 mm in diameter), the skull thickness (5, 6, 7, and 8 mm), the absorption coefficient of the skin (−70%, −60%, . . . , +60%, +70%), and the position of the source position (−1, 0, and +1 mm).

The data obtained from the simulation were parsed using a python script, and were then split into a test set and a training set for use using a machine learning procedure (logistic regression, in the present example), aiming to train the machine learning procedure to determine whether or not the data describes existence of hematoma. The data was preprocessed by removing the mean, scaling to unit variance and performing logistic regression.

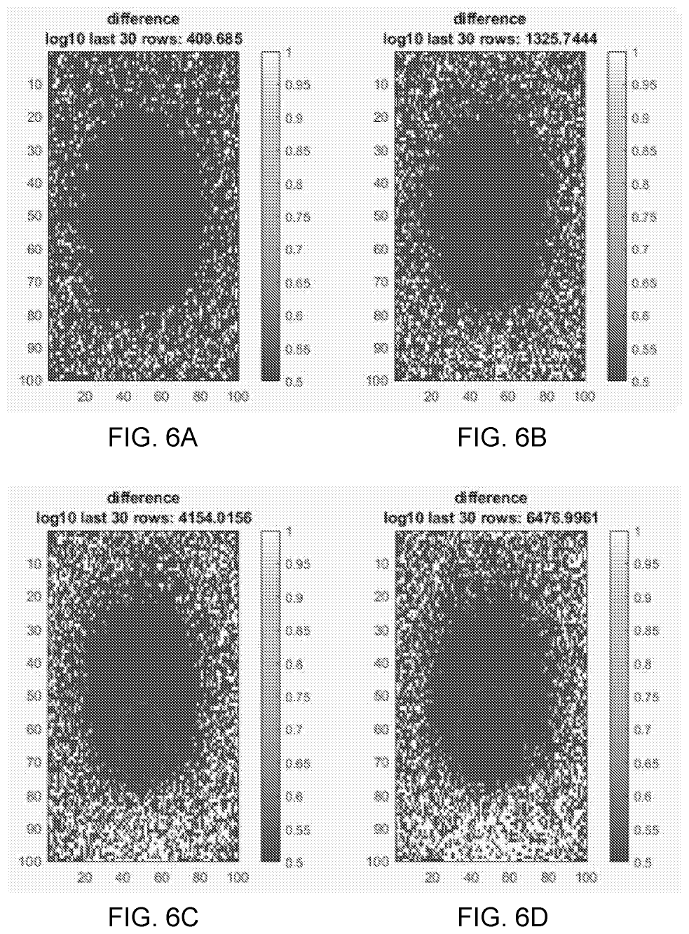

FIGS. 6A-D shows the measured radiation intensity at the detectors. The dotted line shows a hematoma. Since the maximum difference of photon counts was observed at the detectors on a row beneath the source, only data collected by these detectors were used.

The machine learning procedure (logistic regression, in the present example) was applied for each of the NIR bands and each of the radiofrequency bands, separately as well as in combination. For the logistic regression procedure, the procedure described in Pedregosa et al., Scikit-learn: Machine Learning in Python, JMLR 12, pp. 2825-2830, 2011 was used, with the parameters listed in Table 1, below.

TABLE 1

| | |
|---|---|
| penalty | ‘l2’ |
| dual formulation | False |
| tolerance | 0.0001 |
| regularization strength inverse | 1.0 |
| the decision function with an intercept | True |
| scaling parameter for the intercept | 1 |
| weights associated with classes | None |
| seed used by the pseudo random number generator | 42 |
| solver type | ‘warn’ |
| maximum number of iterations for the solver | 100 |
| multi class type | ‘warn’ |
| verbosity | 0 |
| reuse the solution of the previous call | False |
| CPU parallelization | None |
| Elastic-Net mixing parameter | None |

Following the logistic regression, a Receiver Operating Characteristic (ROC) curve was constructed, and an Area Under the ROC Curve (AUC) score was calculated for two wavelengths separately and in combination. The logistic model was used as a binary classifier to estimate the probability of a certain class or event existing such as healthy/sick. The ROC curve is a graphical plot that illustrates the diagnostic ability of a binary classifier system as its discrimination threshold is varied. ROC curves typically feature true positive rate (Sensitivity) on the Y axis, and false positive rate (100-Specificity) on the X axis. This means that the top left corner of the plot is the “ideal” point—a false positive rate of zero, and a true positive rate of one. The AUC is a measure of how well a parameter can distinguish between two classes.

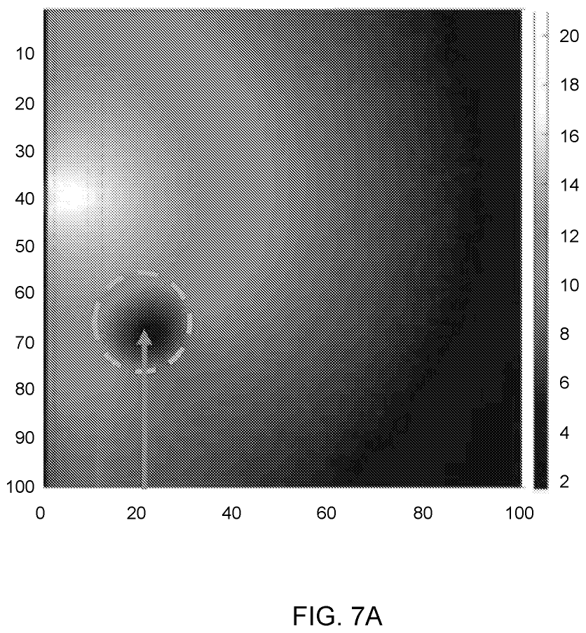

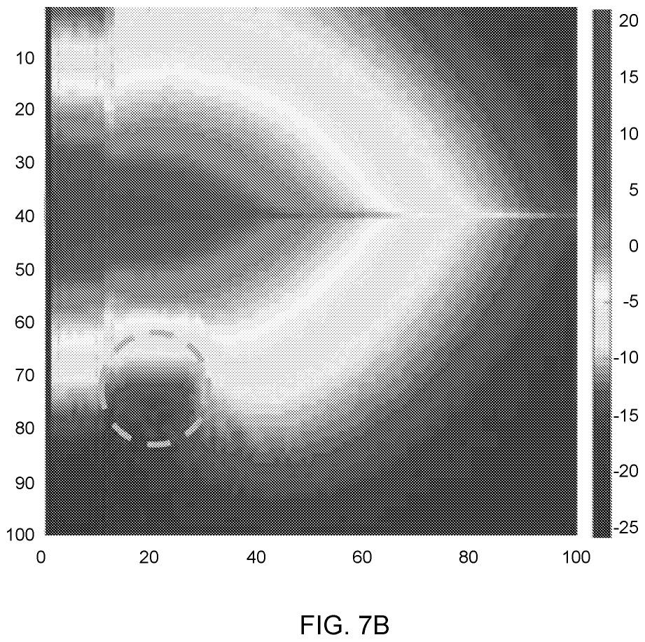

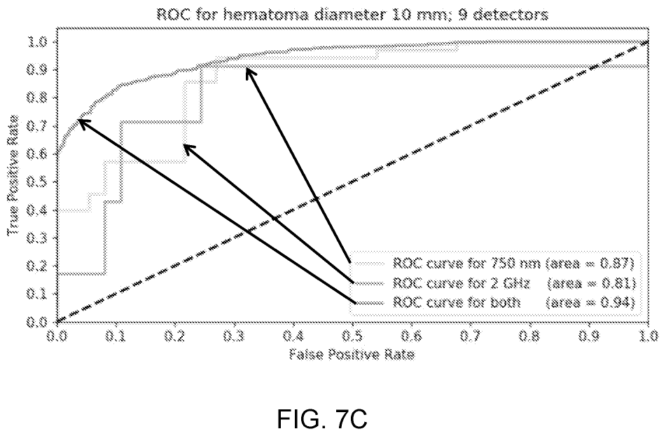

The simulations results are shown in FIGS. 7A-C. FIG. 7A shows a logarithmic based intensity map illustration of optical photons propagation, over the 3D head model, as obtained by simulations for the optical sub-system. The axes X and Y show distances in mm, the color coded scale is the relative photon counts. FIG. 7B shows a logarithmic based intensity map illustration of RF waves propagation over the 3D head model, as obtained by simulations for the sub-optical sub-system. The axes X and Y show distances in mm, the color coded scale is the relative RF quanta counts.

FIG. 7C shows the ROC curve for a simulated hematoma, 10 mm in diameter, using 9 detectors for (i) NIR wavelength of 750 nm (AUC=0.87), (ii) RF of 2 GHz (AUC=0.81), and combination of (i) and (ii) (AUC=0.94). As shown, the ROC curve when using the combination is significantly higher than the ROC curve obtained using only optical radiation and only RF radiation, demonstrating a synergistic effect of the radio-optical sub-system of the present examples.

### Experiments

### Materials and Methods

Gelatin based phantoms were prepared due to the ability to customize their optical properties by incorporating scattering agents (e.g., intralipid/milk), and/or absorbing agents (e.g., India ink, or dye), to modify their electrical and dielectric properties by varying the fraction of gelatin, water content, sugar and salinity. Gelatin based phantoms are also advantageous due to their customizable mechanical properties. In this Example, two types of phantoms were prepared: spherical phantoms and anthropomorphic human head phantoms.

All gelatin phantoms were prepared based on the following procedure:
(i) A mold was constructed in a way which allows, producing 2 hemispheres of outer layer with an empty space for filling with a new portion of gelatin solution into the inner layer.
(ii) Gelatin for the outer layer was melted in distillated water and additional components were added according to a protocol further detailed below.
(iii) Gelatin for the inner layer was melted in distillated water and additional components were added according to the protocol detailed below. Gelatin solution for the inner layer was added after polymerization of the gelatin in the outer layer, following 12 hour incubation at +4° C.
(iv) Following polymerization of both outer and inner layers, the hemispheres were glued together using heated wire.

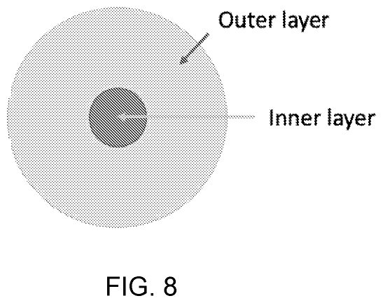

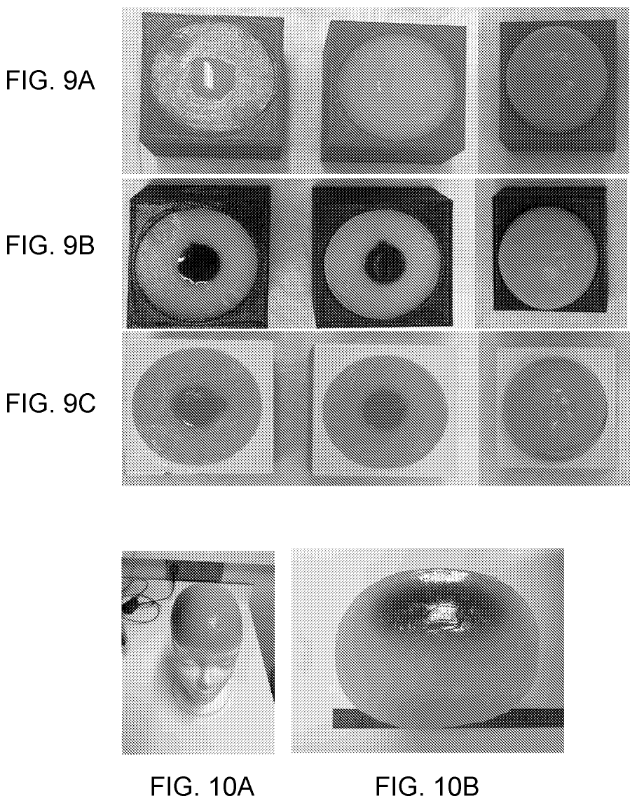

FIG. 8 is a schematic illustration of a bi-layered spherical gelatin phantom, prepared for experiments conducted according to some examples of the present disclosure. The outer diameter was 5.5 cm and the inner diameter was 1.5 cm. Three variants of the bi-layered gelatin spheres were prepared, and are shown in FIGS. 9A-C, where FIG. 9A shows a phantom with low radio-optical contrast, mimicking a tissue without, or with low levels of, hemoglobin, FIG. 9B shows a phantom with high radio-optical contrast, mimicking blood (e.g., hematoma), and FIG. 9C shows a phantom with high radio contrast and low optical contrast, mimicking CSF.

The compositions used for fabricating the inner layer and outer layer of each of the phantoms shown in FIGS. 9A-C are summarized in Table 2, below.

| | spherical | | spherical | | spherical | |
|---|---|---|---|---|---|---|
| | tissue | | hematoma | | CSF | |
| PHANTOM | (FIG. 9A) | | (FIG. 9B) | | (FIG. 9C) | |
| layer | inner | outer | inner | outer | inner | outer |
| fish gelatin | 10% | 10% | 5% | 10% | 5% | 10% |
| milk | 0.3% | 0.3% | 0.3% | 0.3% | 0.3% | 0.3% |
| red ink/100 ml | 10 μl | 10 μl | 10 μl | 10 μl | 30 μl | 10 μl |
| blue ink/100 ml | — | — | 10 μl | — | — | — |
| green ink/100 ml | — | — | 10 μl | — | — | — |
| black ink/100 ml | — | — | 10 μl | — | — | — |
| sodium chloride | 0.5% | 0.5% | 0.9% | 0.5% | 0.9% | 0.5% |

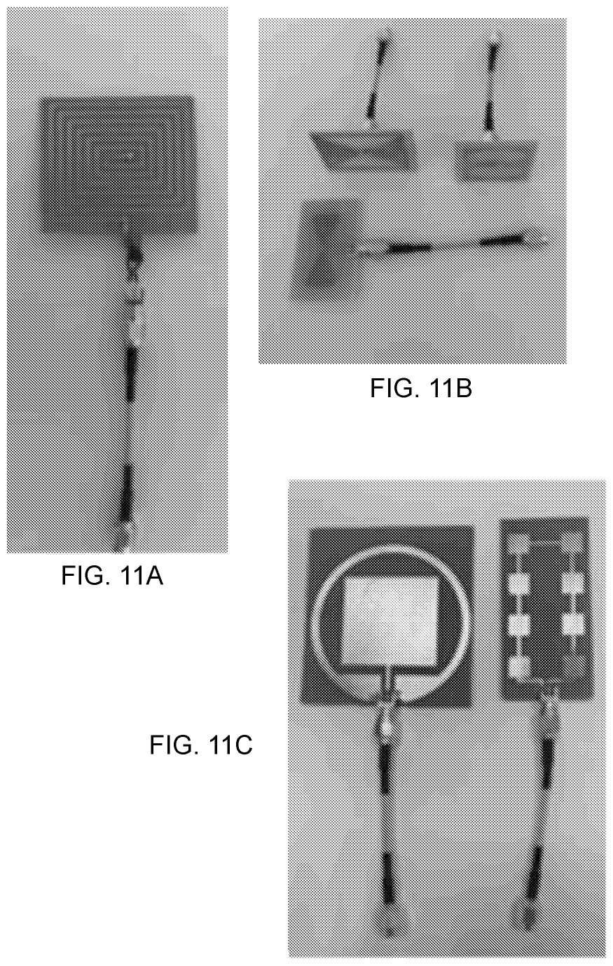

FIGS. 10A and 10B are images of an anthropomorphic human head gelatin phantom with hematoma, prepared for experiments conducted according to some examples of the present disclosure. In this phantom, the outer layer included fish gelatin 10%, milk 0.3%, red ink 10 μml per 100 ml, and sodium chloride 0.5%, and the inner layer included fish gelatin 5%, milk 0.3%, red ink 10 μml per 100 ml, blue ink 10 μml per 100 ml, green ink 10 μl per 100 ml, black ink 10 μl per 100 ml, and sodium chloride 0.9%.

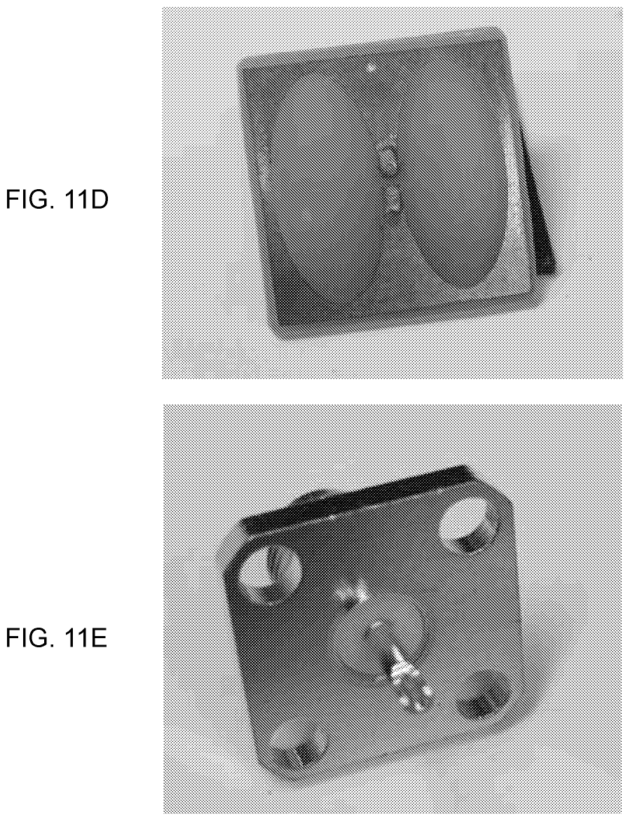

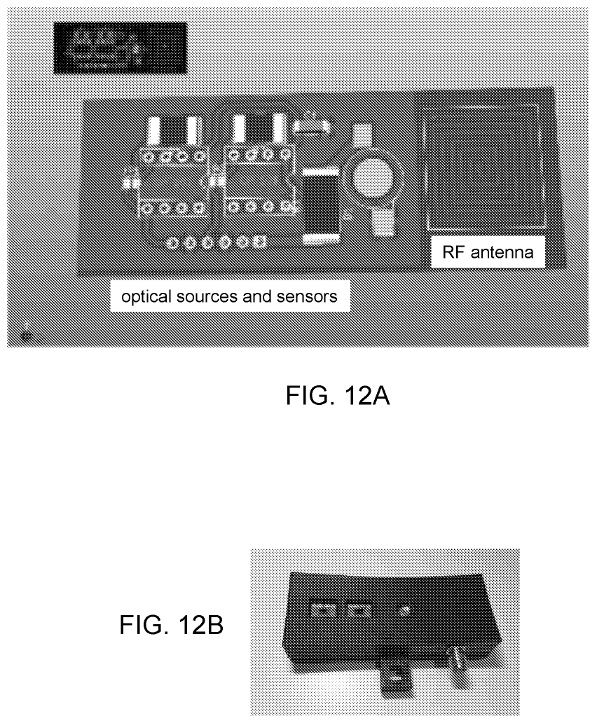

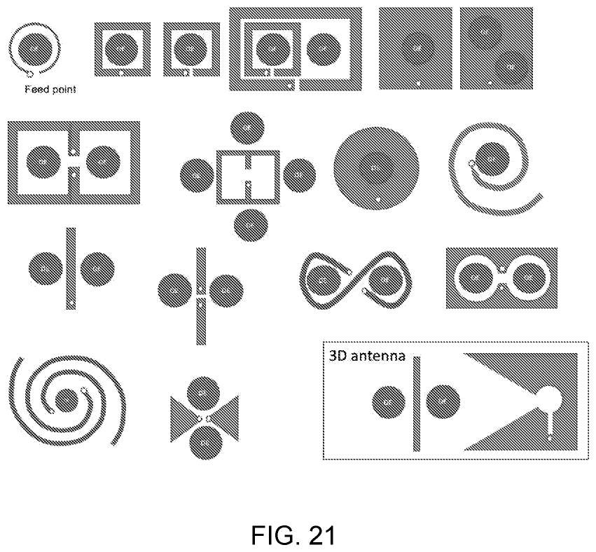

A dedicated setup for simultaneous testing of both radio and optical modalities was developed and used. The radiation sources (antennas) and the receivers were developed in parallel with the computer simulation process. Images of some working examples of various types of antennas are shown in FIGS. 11A-E. Images of a radio-optical sensor prepared according to some examples of the present disclosure are provided in FIGS. 12A-B. Configurations of sub-optical antennas and optical sources and/or detectors, contemplated according to some examples of the present disclosure are illustrated in FIG. 21. In FIG. 21, OE refers to the location of the optical sources and/or detectors, and feed point refers to the component of the antenna which feeds the sub-optical waves to the antenna.

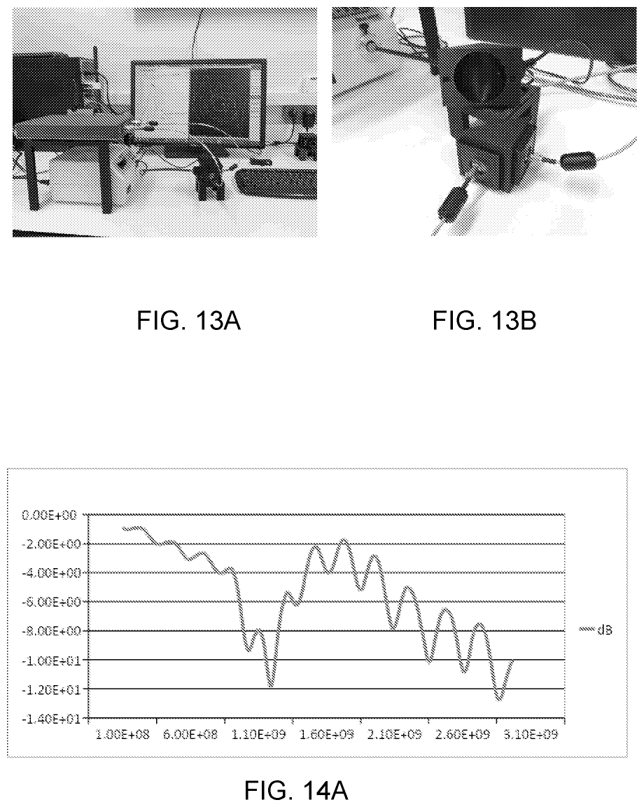

The setup for the experiments with the spherical phantoms included a Fiber-Lite MI-150 High intensity illuminator as a light source and USB2000 OceanOptics spectrometer as light sensitive instrument. In addition to the optical modality, a Copper Mountain M5090 Network Analyzer 300 kHz-8.5 Ghz was used for S11, S12 amplitude and phase measurements. The absorbance of the three spheres was measured using Ocean View software. The red sphere (FIG. 9A) was used for absorbance calibration. Images of the experimental setup is shown in FIGS. 13A and 13B. For the anthropomorphic phantom the network analyzer was used with a dual band transmitter and receiver.

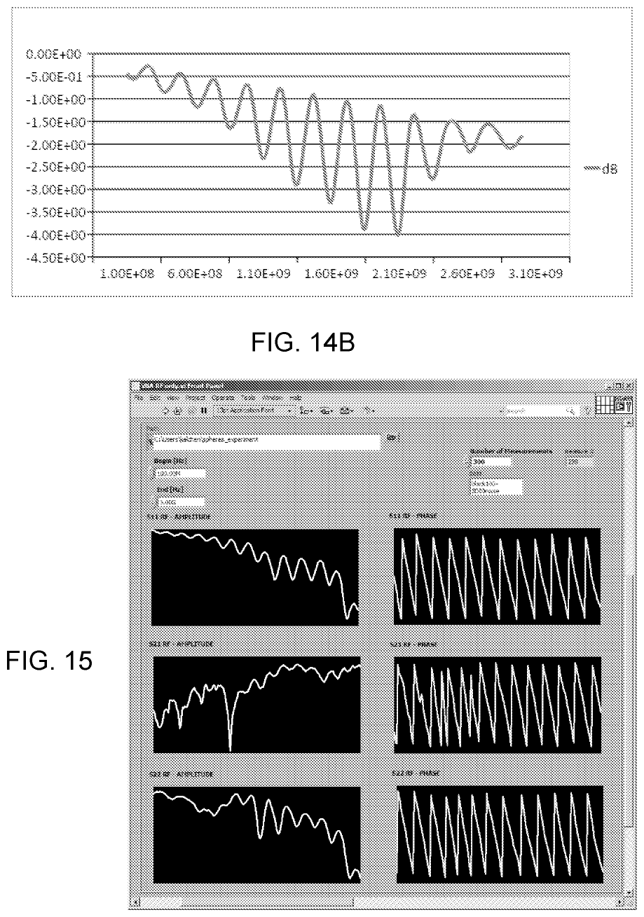

The radiofrequency parameter S11 represents the amount of power that is reflected off the antenna, and is therefore oftentimes referred to as the reflection coefficient. When S11=0 dB, then all the power is reflected from the antenna and the antenna does not radiate. A value of, e.g., −10 dB for S11 means that if the antenna is provided with power of 3 dB of power, −7 dB are reflected. The remainder of the power was accepted by or delivered to the antenna. This accepted power is either radiated or absorbed as losses within the antenna. Since antennas are typically designed to be low loss, ideally the majority of the power delivered to the antenna is radiated. FIG. 14A is a graph showing the S11 parameter of a butterfly antenna, and FIG. 14B is a graph showing the S11 parameter of a pin antenna. Both types of the antennas shown in FIGS. 11D and 11E were tested. In the present Example, the antenna shown in FIG. 11D was used for the data collection from the sphere phantom.

FIG. 15 is an example screen image showing data acquisition of RF data, for 300measurements using a spherical phantom.

The acquired data was fed to a machine learning procedure. In these experiments, two types of machine learning procedures were tested: linear regression and extreme gradient boosting. For classification purposes, amplitude signals of S11, S21 of 100 Mhz and 1500 Mhz frequencies were obtained. For the optical measurements, central wavelengths of 765 nm and 830 nm were selected. The dataset was split according to a training/test ratio of 80/20. The machine learning procedure was applied to provide binary classification. The classifiers were trained on three different datasets: (i) only RF data, (ii) only optical data, and combination of (i) and (ii).

For the logistic regression, the following parameters were used: regularization 12, liblinear solver, and maximum number of iterations 100. For the extreme gradient boosting, the following parameters were used: boosting type—Gradient Boosting Decision Tree, objective—binary log loss classification, 100 boosting iterations, learning rate 0.1, number of leaves 31, maximum depth for tree model—no limit, minimum data in leaf 20, L1, L2 regularizes=0.

### Results

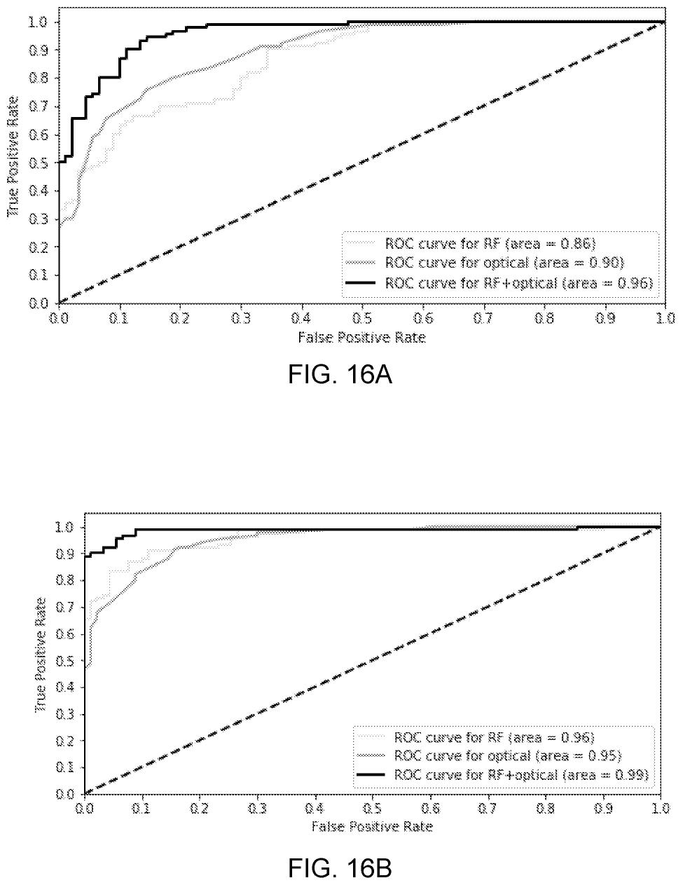

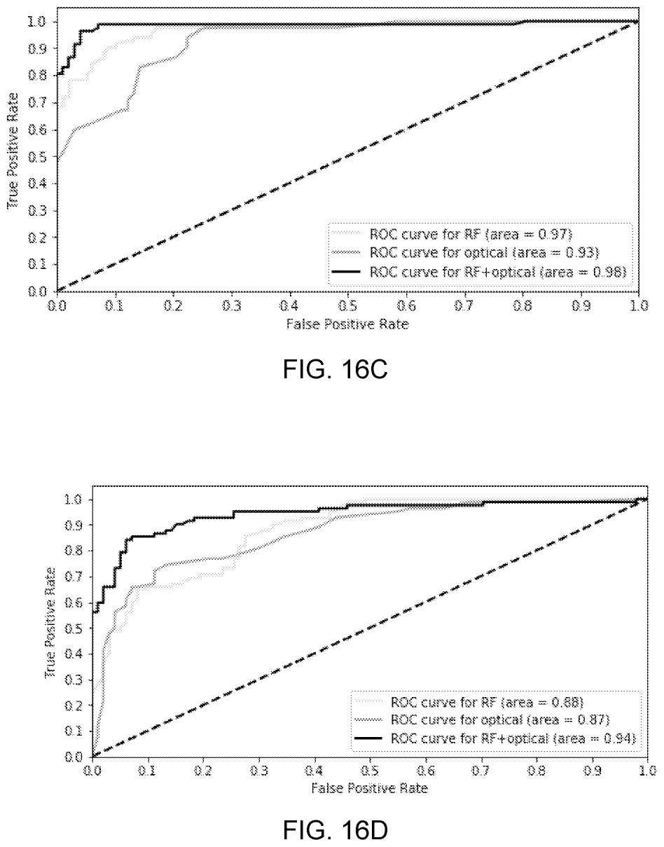

FIGS. 16A-D show ROC curves graphs for the butterfly antenna with RF of 100 Mhz, and central NIR wavelengths of 765 nm and 830 nm. Shown are results obtained using the logistic regression (LG) procedure (FIGS. 16A-B) and extreme gradient boosting (EGB) procedure (FIGS. 16C-D), for blank vs blood classification (FIGS. 16A and 16C), and blank vs CSF classification (FIGS. 16B and 16D). The AUC values for the ROC curves shown in FIGS. 16A-D are summarized in Table 3, below.

TABLE 3

| | |
|---|---|
| blank vs blood | blank vs CSF |

| | LR | EGB | LR | EGB |
|---|---|---|---|---|
| AUC based on signal 20 | 0.90 | 0.93 | 0.95 | 0.93 |
| AUC based on signal 26 | 0.86 | 0.97 | 0.96 | 0.88 |
| AUC based on both | 0.96 | 0.98 | 0.99 | 0.94 |
| signals 20 and 26 | | | | |

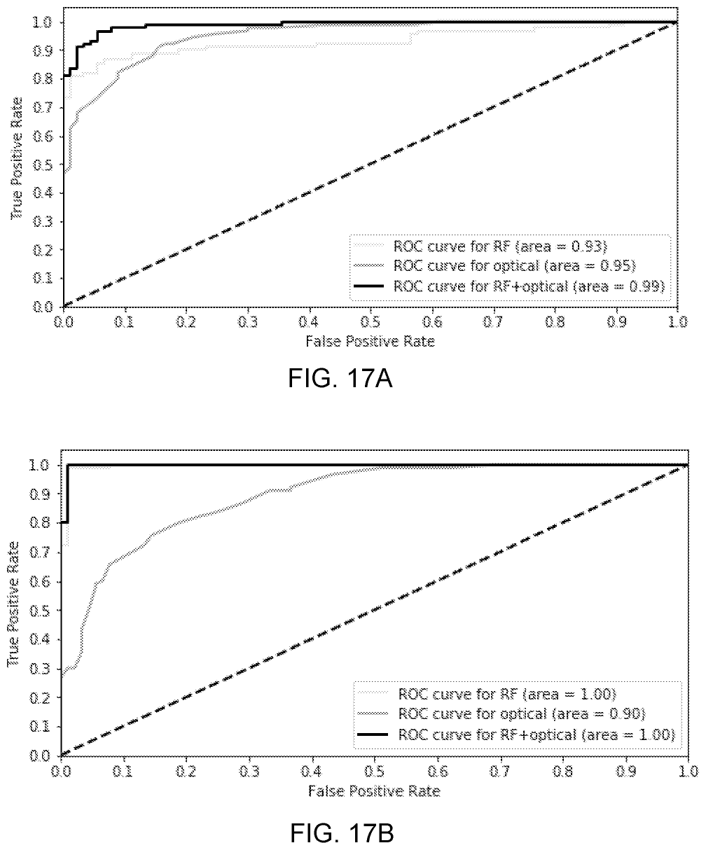

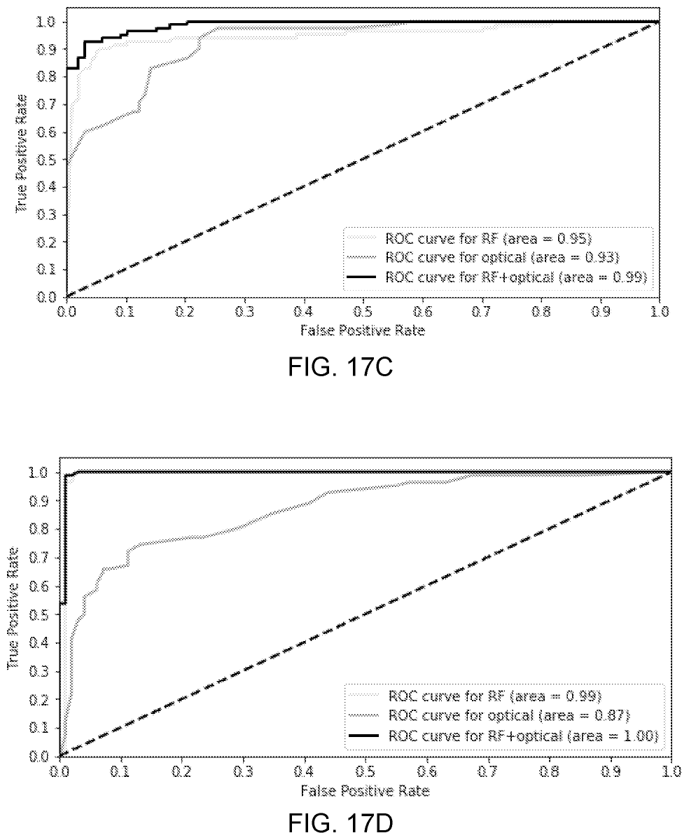

FIGS. 17A-D show ROC curves graphs for the butterfly antenna with RF of 1500 Mhz, and central NIR wavelengths of 765 nm and 830 nm. Shown are results obtained using the LG procedure (FIGS. 17A-B) and EGB procedure (FIGS. 17C-D), for blank vs blood classification (FIGS. 17A and 17C), and blank vs CSF classification (FIGS. 17B and 17D). The AUC values for the ROC curves shown in FIGS. 17A-D are summarized in Table 4, below.

TABLE 4

| | |
|---|---|
| blank vs blood | blank vs CSF |

| | LR | EGB | LR | EGB |
|---|---|---|---|---|
| AUC based on signal 20 | 0.95 | 0.93 | 0.90 | 0.87 |
| AUC based on signal 26 | 0.93 | 0.95 | 1 | 0.99 |
| AUC based on both | 0.99 | 0.99 | 1 | 1 |
| signals 20 and 26 | | | | |

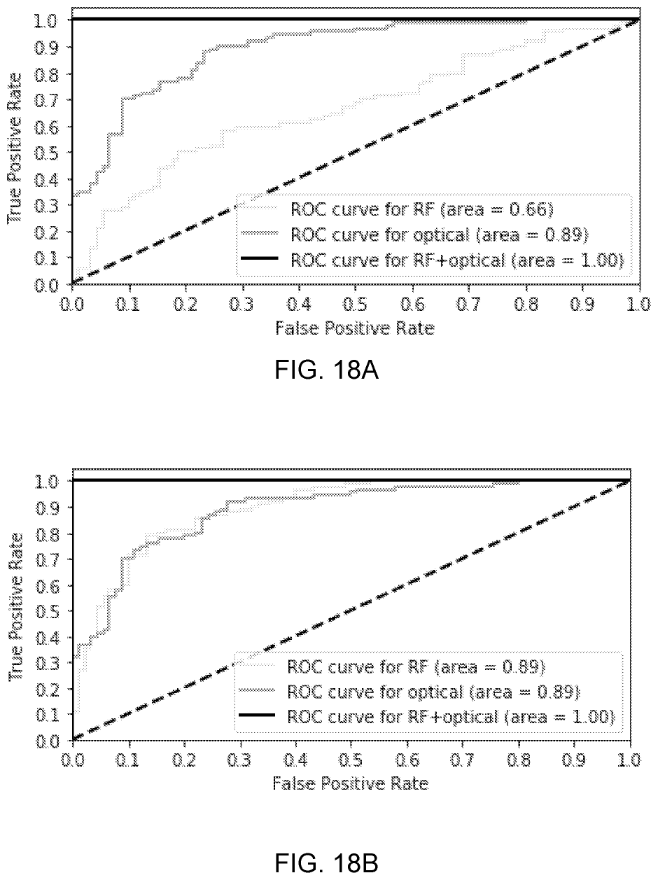

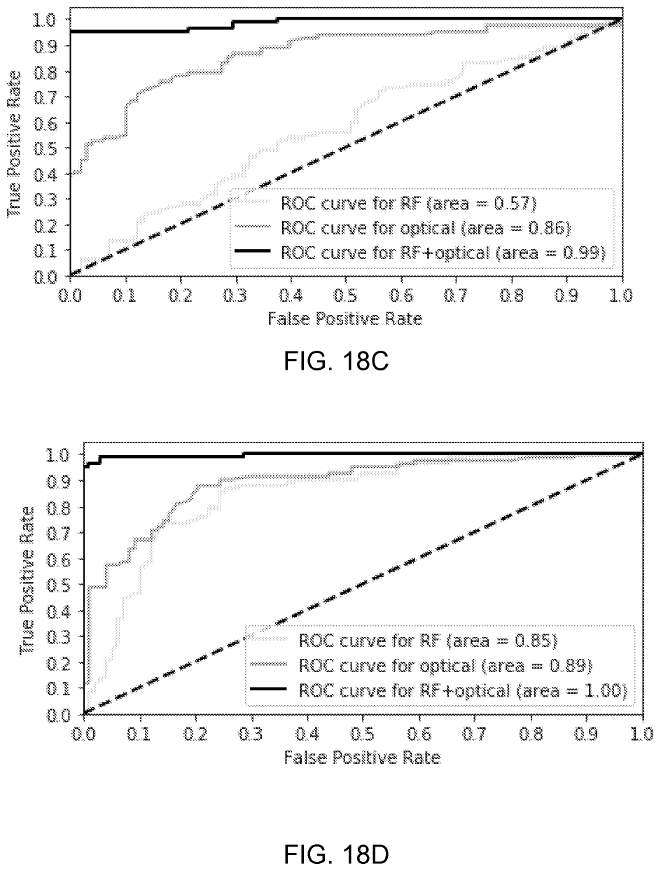

FIGS. 18A-D show ROC curves graphs for the pin antenna with RF of 100 Mhz, and central NIR wavelengths of 765 nm and 830 nm. Shown are results obtained using the LG procedure (FIGS. 18A-B) and EGB procedure (FIGS. 18C-D), for blank vs blood classification (FIGS. 18A and 18C), and blank vs CSF classification (FIGS. 18B and 18D). The AUC values for the ROC curves shown in FIGS. 18A-D are summarized in Table 5, below.

TABLE 5

| | |
|---|---|
| blank vs blood | blank vs CSF |

| | LR | EGB | LR | EGB |
|---|---|---|---|---|
| AUC based on signal 20 | 0.89 | 0.86 | 0.89 | 0.89 |
| AUC based on signal 26 | 0.66 | 0.57 | 0.89 | 0.85 |
| AUC based on both | 1 | 0.99 | 1 | 1 |
| signals 20 and 26 | | | | |

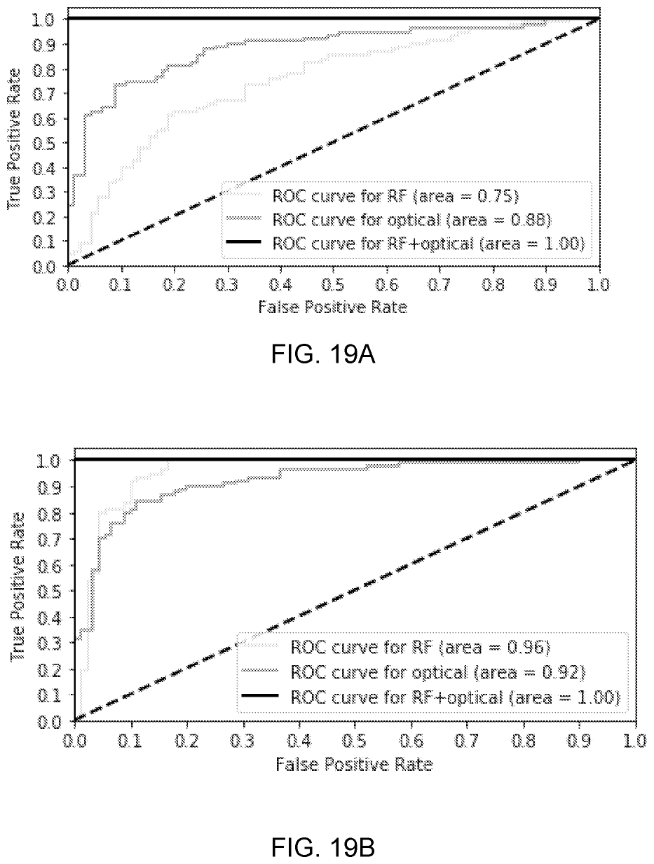

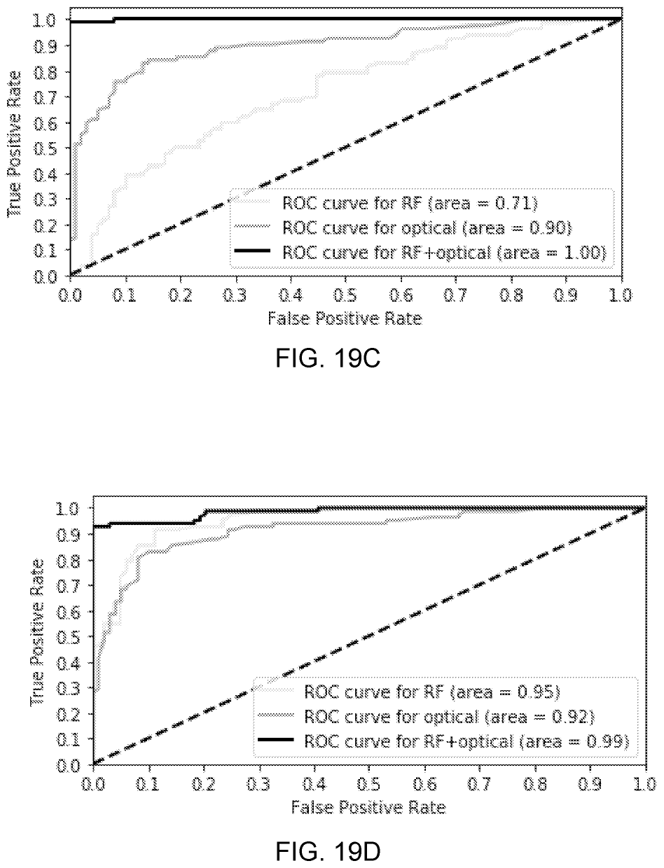

FIGS. 19A-D show ROC curves graphs for the pin antenna with RF of 1500 Mhz, and central NIR wavelengths of 765 nm and 830 nm. Shown are results obtained using the LG procedure (FIGS. 19A-B) and EGB procedure (FIGS. 19C-D), for blank vs blood classification (FIGS. 19A and 19C), and blank vs CSF classification (FIGS. 19B and 19D). The AUC values for the ROC curves shown in FIGS. 19A-D are summarized in Table 6, below.

TABLE 6

| | |
|---|---|
| blank vs blood | blank vs CSF |

| | LR | EGB | LR | EGB |
|---|---|---|---|---|
| AUC based on signal 20 | 0.88 | 0.90 | 0.92 | 0.92 |
| AUC based on signal 26 | 0.75 | 0.71 | 0.96 | 0.95 |
| AUC based on both | 1 | 1 | 1 | 0.99 |
| signals 20 and 26 | | | | |

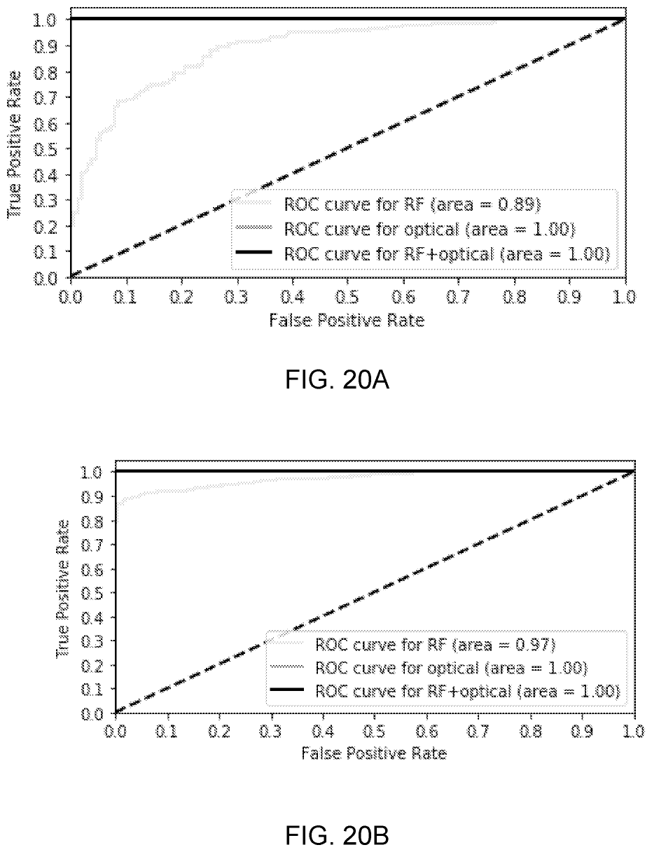

FIGS. 20A and 20B show ROC curves graphs measured for the brain phantom with RF of 100 Mhz (FIG. 20A) and 1500 Mhz (FIG. 20B). Shown are results obtained using the LG procedure. The AUC values for the ROC curves shown in FIGS. 20A-B are summarized in Table 7, below.

TABLE 7

| | 100 Mhz | 1500 Mhz |
|---|---|---|
| AUC based on signal 20 | 1 | 1 |
| AUC based on signal 26 | 0.89 | 0.97 |
| AUC based on both signals 20 and 26 | 1 | 1 |

The AUC values for optical and RF+optical modality are maximal (=1) and therefore they overlap.

### Prototype Module and System

A prototype module was designed, according to some examples of the present disclosure, based on the configuration shown in FIGS. 22A and 22B.

The prototype module is assembled from two carrier substrates 102 and 122. FIGS. 23A-D illustrate a planar view (FIGS. 23A and 23C) and an isometric view (FIGS. 23B and 23D) of a front side (FIGS. 23C-D) and a back side (FIGS. 23A-B) of a first carrier substrate 102 of the prototype module (also shown at 102 in FIG. 22A). The carrier substrate 102 is formed with a through-hole 112 at or near the center of the substrate, for receiving a light emitting element of system 30 (not shown, see FIG. 24B). The carrier substrate 102 may optionally and preferably be formed with additional openings 114 for receiving other electronic components of optical sub-system 14 (not shown, see FIGS. 24A and 24B).

FIG. 24A illustrates a planar view of a back side of a second carrier substrate 122 of the prototype module, and FIG. 24B illustrates an isometric view of a front side of second carrier substrate 122. Various electronic components 124 of optical sub-system 14 (such as, but not limited to, electronic chips, connectors and the like) are mounted on the front side of second carrier substrate 122, with their contacts on the back side thereof. A light emitting element 126 is also mounted at or near the center of the front side second carrier substrate 122.

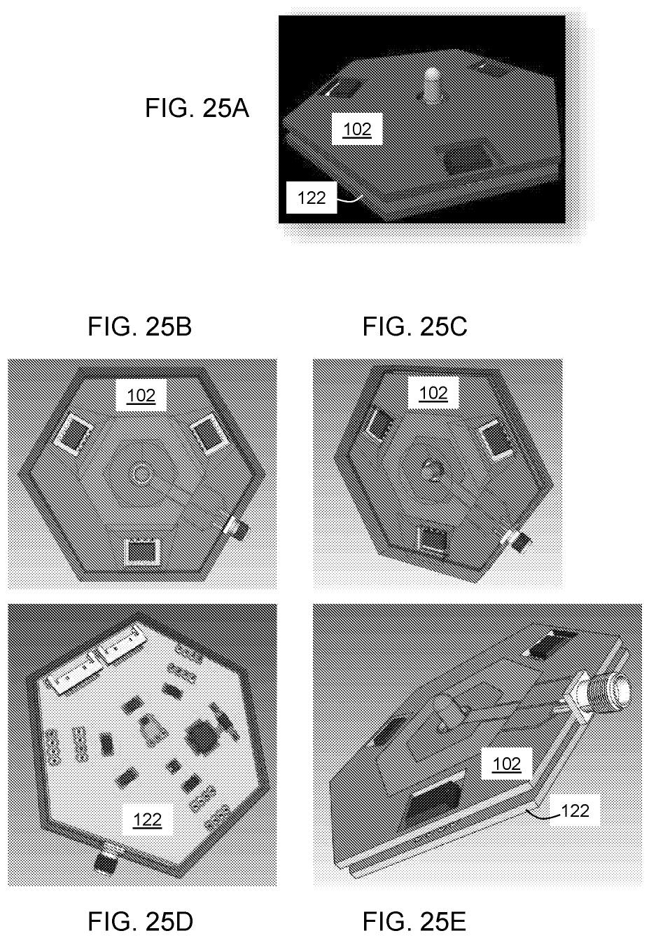

FIGS. 25A-E illustrate the assembled module from various viewpoints.

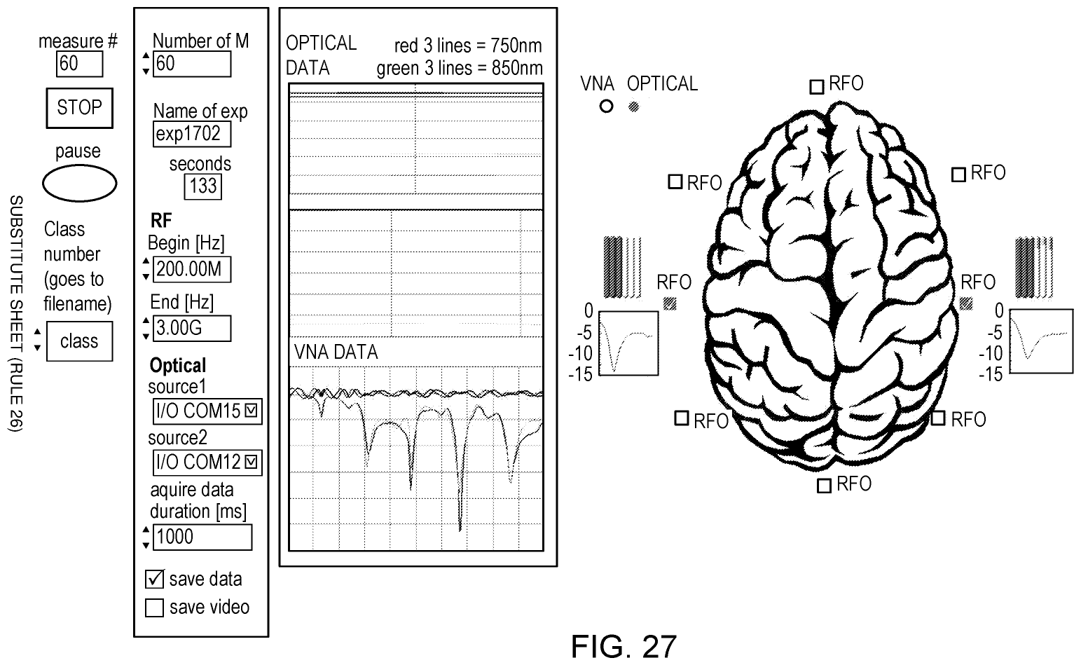

FIG. 27 illustrates a representative example of a graphical user interface (GUI) that is generated by the data processor of a prototype system prepared according to some examples of the present disclosure.

### Exemplary Synchronization Protocols

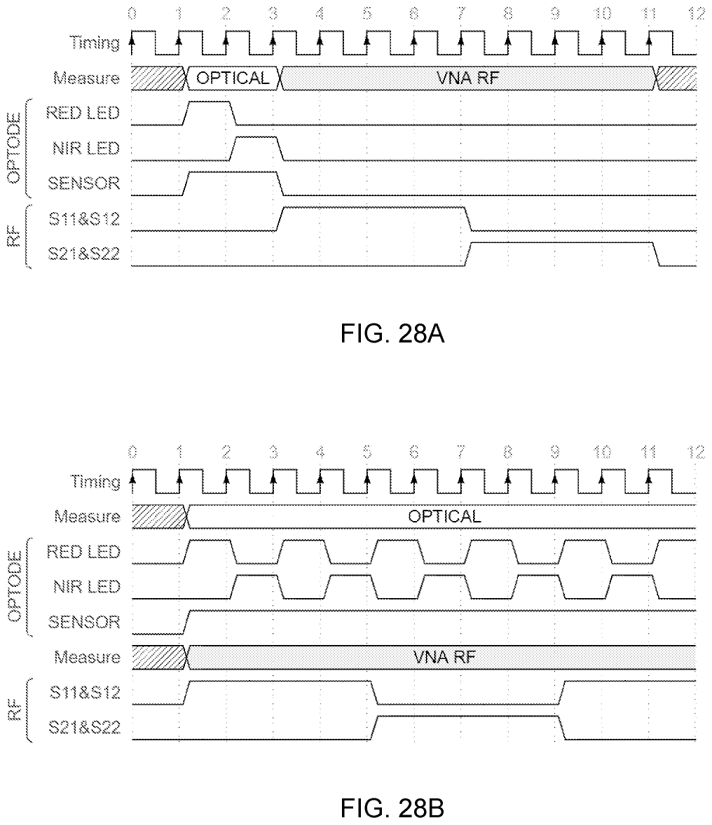

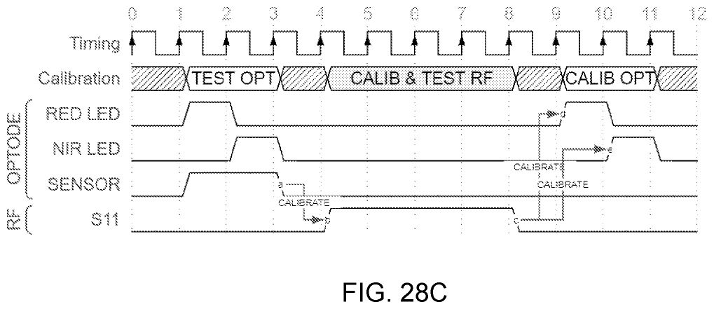

FIGS. 28A-C illustrate exemplary synchronization protocols suitable for some examples of the present disclosure. FIG. 28A illustrates a synchronization protocol suitable for operating the optical sub-system and radio transceiver sub-system in sequential mode, FIG. 28B illustrates a synchronization protocol suitable for operating the optical sub-system and radio transceiver sub-system in continuous mode, and FIG. 28C illustrates a synchronization protocol suitable for executing mutual calibration among the optical sub-system and the radio transceiver sub-system.

Although the invention has been described in conjunction with specific examples thereof, it is evident that many alternatives, modifications and variations will be apparent to those skilled in the art. Accordingly, it is intended to embrace all such alternatives, modifications and variations that fall within the spirit and broad scope of the appended claims.

### Additional Examples of the Disclosed Technology

In view of the above-described examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example A1. A system for detecting and/or assessing a subdural hematoma, comprising: a wearable structure, configured to be worn on a head of a subject; an optical sub-system, mounted on the wearable structure and being configured for emitting light towards a predetermined location in relation to the wearable structure, the predetermined location coinciding with a predetermined area of the head of the subject, and sensing the emitted light returning from the predetermined location, wherein the optical sub-system being further configured for generating a respective set of signals responsively to interactions of the emitted light with the brain of the subject; a radio transceiver sub-system configured for emitting sub-optical radiation and generating a signal responsively to an interaction of the radiation with the brain of the subject; and a data processor configured to: analyze the signals of the optical sub-system and the radio transceiver sub-system, and detect and/or assess a subdural hematoma of the subject based on the analysis, wherein, while the optical sub-system is mounted on the wearable structure, the predetermined location is adjustable.

Example A2. The system of any example herein, particularly example A1, further comprising: at least one actuator; and a controller, wherein the at least one actuator is configured, responsive to the controller, for adjusting the predetermined location.

Example A3. The system of any example herein, particularly example A2, wherein the optical sub-system comprises at least one light source being configured for the emitting of the light and at least one optical sensing element being configured for the sensing of the light, and wherein the at least one actuator is configured for adjusting a position of the at least one light source and/or the at least one optical sensing element in relation to the wearable structure to thereby adjust the predetermined location.

Example A4. The system of any example herein, particularly example A3, wherein the optical sub-system comprises a plurality of optical sensing elements, the at least one actuator configured for adjusting the positions of the plurality of optical sensing elements independently from each other.

Example A5. The system of any example herein, particularly any of examples A3-A4, wherein the wearable structure comprises a platform and a static structure, the at least one optical sensing element being mounted on the platform, wherein the at least one actuator is configured, responsive to the controller, to move the platform in respective to the static structure.

Example A6. The system of any example herein, particularly example A5, wherein the at least one light source is mounted on the platform.

Example A7. The system of any example herein, particularly any one of examples A5-A6, wherein the radio transceiver sub-system is mounted on the platform.

Example A8. The system of any example herein, particularly example A1, wherein the optical sub-system comprises a plurality of light sources, each configured for emitting light in a respective direction, wherein the predetermined location is adjusted by selecting a respective one of the plurality of light sources.

Example A9. The system of any example herein, particularly example A1 or example A8, wherein the optical sub-system comprises a plurality of sets of light sensors, each set of light sensors configured for sensing light from a respective direction, wherein the predetermined location is adjusted by selecting a respective one of the plurality of sets of light sensors.

Example A10. The system of any example herein, particularly example A1, wherein the radio transceiver sub-system comprises a plurality of antennas, each configured for emitting sub-optical radiation in a respective direction, wherein the predetermined location is adjusted by selecting a respective one of the plurality of antennas.

Example A11. The system of any example herein, particularly example A1, further comprising a controller, the optical sub-system and the radio transceiver sub-system operated by the controller, wherein, based on the analysis of the signals of the optical sub-system, the data processor is configured to generate an initial detection notification, and
wherein the controller is configured to control the radio transceiver sub-system to emit the sub-optical radiation responsive to the generated initial detection notification.

Example A12. The system of any example herein, particularly example A1 ,further comprising a controller, the optical sub-system and the radio transceiver sub-system operated by the controller, wherein, based on the analysis of the signals of the radio transceiver sub-system, the data processor is configured to generate an initial detection notification, and wherein the controller is configured to control the optical sub-system to emit the light responsive to the generated initial detection notification.

Example A13. The system of any example herein, particularly example A1, wherein the data processor comprises a predetermined analysis model configured to receive the signals of the optical sub-system and the radio transceiver sub-system, the analysis being responsive to the predetermined analysis model.

Example A14. The system of any example herein, particularly example A1, wherein the predetermined analysis model comprises a convolutional neural network.

Example A15. The system of any example herein, particularly example A1, further comprising one or more additional sensors selected from the group consisting of one or more ultrasound transceivers, one or more electrical impedance sensors, one or more electroencephalogram (EEG) sensors, one or more electromyography (EMG) sensors and one or more temperature sensors, wherein the data processor is configured to receive from the one or more additional sensors information regarding a respective attribute of the subject.

Example A16. The system of any example herein, particularly example A15, further comprising a controller, the one or more additional sensors operated by the controller, wherein the controller is configured to operate the one or more additional sensors responsive to a respective output of the data processor.

Example A17. The system of any example herein, particularly any one of examples A1-A16, wherein the sub-optical radiation comprises a plurality of frequencies, a first of the plurality of frequencies being at least 10 times a second of the plurality of frequencies.

Example A18. The system of any example herein, particularly example A17, wherein the first of the plurality of frequencies is at least 100 times a second of the plurality of frequencies.

Example A19. The system of any example herein, particularly example A18, wherein the first of the plurality of frequencies is at least 1,000 times the second of the plurality of frequencies.

Example A20. The system of any example herein, particularly any one of examples A1-A19, wherein the data processor is configured for delineating a boundary within the brain at least partially encompassing a region having functional and/or structural properties that are different from regions outside the boundary.

Example A21. The system of any example herein, particularly any one of examples A1-A20, wherein the data processor is configured to analyze a signal generated by the optical sub-system and a signal generated by the radio transceiver sub-system, separately for each one of a plurality of predetermined locations.

Example A22. The system of any example herein, particularly any one of examples A1-A20, wherein the data processor is configured to provide functional and/or structural information for each of a plurality of predetermined locations.

Example A23. The system of any example herein, particularly example A22, wherein the data processor is configured to: calculate a respective signal-to-noise ratio for each of the plurality of predetermined locations; and select one of the plurality of predetermined location for further light emission based on the calculated signal-to-noise ratios.

Example A24. The system of any example herein, particularly any one of examples A1-A23, wherein the data processor is configured to analyze the signals to determine displacements of at least one of the optical and the radio transceiver sub-systems.

Example A25. The system of any example herein, particularly any one of examples A1-A24, wherein the data processor is configured to analyze signals received from the optical sub-system to determine proximity between the wearable structure and the head.

Example A26. The system of any example herein, particularly any one of examples A1-A25, wherein the data processor is configured to analyze signals received from the radio transceiver sub-system to determine whether the wearable structure is mounted on a living head.

Example A27. The system of any example herein, particularly example A26, wherein the data processor is configured to analyze signals received from the radio transceiver sub-system to determine dielectric properties of the tissue and to transmit control signals to the optical sub-system based on the determined dielectric properties.

Example A28. The system of any example herein, particularly any one of examples A1-A27, comprising a communication device configured for transmitting the functional and/or structural information to a remote monitoring location. Example B1. A system for detecting and/or assessing a subdural hematoma, comprising: a wearable structure, configured to be worn on a head of the subject; an optical sub-system, movably mounted on the wearable structure and being configured for emitting light while assuming a set of different positions relative to the wearable structure and for generating a respective set of signals responsively to interactions of the light with the brain; a radio transceiver sub-system configured for emitting sub-optical radiation and generating a signal responsively to an interaction of the radiation with the brain; and a data processor configured to analyze the signals and to provide functional and/or structural information describing the brain based on the analysis.

Example B2. The system of any example herein, particularly example B1, the optical sub-system comprises a plurality of optical sensing elements for sensing the light.

Example B3. The system of any example herein, particularly example B2, the plurality of optical sensing elements are movable independently from each other.

Example B4. The system of any example herein, particularly example B2, the plurality of optical sensing elements are movable synchronously with each other.

Example B5. The system of any example herein, particularly example B4, the wearable structure comprises a platform movable with respect to a static structure, wherein the plurality of optical sensing elements are mounted on the platform.

Example B6. The system of any example herein, particularly example B5, the optical sub-system comprises a light sensor which is also mounted on the platform.

Example B7. The system of any example herein, particularly example B5, the radio transceiver sub-system is also mounted on the platform.

Example B8. The system of any example herein, particularly any of examples B1-B7, the data processor is configured for delineating a boundary within the brain at least partially encompassing a region having functional and/or structural properties that are different from regions outside the boundary.

Example B9. The system of any example herein, particularly any one of examples B1-B8, the data processor is configured for applying a machine learning procedure for the analysis.

Example B10. The system of any example herein, particularly any one of examples B1-B9, the data processor is configured to analyze a signal generated by the optical sub-system and a signal generated by the radio transceiver sub-system, separately for each one of the different positions.

Example B11. The system of any example herein, particularly any one of examples B1-B9, the system comprises a controller for controlling the optical sub-system to assume each of the positions.

Example B12. The system of any example herein, particularly example B11, the controller is configured for transmitting to the data processor information pertaining to the position, wherein the data processor is configured to the provide functional and/or structural information also based on the positions.

Example B13. The system of any example herein, particularly any one of examples B11-B12, the data processor is configured to calculate signal-to-noise ratio for each of the positions, and to instruct the controller to control the optical sub-system to assume a position based on the calculated signal-to-noise ratio.

Example B14. The system of any example herein, particularly any one of examples B11-B13, the data processor is configured to analyze the signals to determine displacements of at least one of the optical and the radio transceiver sub-systems, and to issue an alert, or instruct the controller to select a new measurement mode or to control the optical sub-system to assume a position based on the calculated displacements.

Example B15. The system of any example herein, particularly any one of examples B1-B14, the data processor is configured to analyze signals received from the optical sub-system to determine proximity between the wearable structure and the head.

Example B16. The system of any example herein, particularly any one of examples B1-B15, the data processor is configured to analyze signals received from the radio transceiver sub-system to determine whether the wearable structure is mounted on a living head.

Example B17. The system of any example herein, particularly example B16, the data processor is configured to analyze signals received from the radio transceiver sub-system to determine dielectric properties of the tissue and to transmit control signals to the optical sub-system based on the determined dielectric properties.

Example B18. The system of any example herein, particularly any one of examples B1-B17, the optical sub-system and the radio transceiver sub-system are configured to operate intermittently.

Example B19. The system of any example herein, particularly any one of examples B1-B17, the optical sub-system and the radio transceiver sub-system are configured to operate simultaneously.

Example B20. The system of any example herein, particularly any one of examples B1-B19, the system comprises a communication device configured for transmitting the functional and/or structural information to a remote monitoring location.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### References

[1] Yang M, Yang Z, Yuan T, Feng W and Wang P (2019) A Systemic Review of Functional Near-Infrared Spectroscopy for Stroke: Current Application and Future Directions. Front. Neurol. 10:58. doi: 10.3389/fneur.2019.00058

[2] Candefjord, S., Winges, J., Malik, A. A. et al. Med Biol Eng Comput (2017) 55:1177. https://doiDOTorg/10.1007/s11517-016-1578-6

[3] Leiming Yu, Fanny Nina-Paravecino, David Kaeli, Qianqian Fang, “Scalable and massively parallel Monte Carlo photon transport simulations for heterogeneous computing platforms,” J. Biomed. Opt. 23 (1), 010504 (2018).

[4] M A Ansari et al “Skull and cerebrospinal fluid effects on microwave radiation propagation in human brain”. J. Phys. D: Appl. Phys. 50 495401

[5] Scikit learn documentation https://scikit-learnDOTorg

[6] Fawcett, Tom (2006). “An Introduction to ROC Analysis”. Pattern Recognition Letters. 27 (8): 861-874. doi: 10.1016/j.patrec.2005.10.010

[7] H. Akarçay, S. Preisser, M. Frenz, and J. Rička, “Determining the optical properties of a gelatin-TiO2 phantom at 780 nm,” Biomed. Opt. Express 3, 418-434 (2012).

[8] Joachimowicz, N.; Duchêne, B.; Conessa, C.; Meyer, O. Anthropomorphic Breast and Head Phantoms for Microwave Imaging. Diagnostics 2018, 8, 85.

[9] http://wwwDOTantenna-theory.com/definitions/sparameters.php

[10] https://lightgbmDOTreadthedocs.io/en/latest/

1. A system for detecting and/or assessing a subdural hematoma, comprising:
a wearable structure, configured to be worn on a head of a subject;
an optical sub-system, mounted on the wearable structure and being configured for emitting light towards a predetermined location in relation to the wearable structure, the predetermined location coinciding with a predetermined area of the head of the subject, and sensing the emitted light returning from the predetermined location, wherein the optical sub-system being further configured for generating a respective set of signals responsively to interactions of the emitted light with skull-contained matter of the subject;
a radio transceiver sub-system configured for emitting sub-optical radiation and generating a signal responsively to an interaction of the radiation with the skull-contained matter of the subject; and
a data processor configured to:
analyze the signals of the optical sub-system and the radio transceiver sub-system, and
detect and/or assess a subdural hematoma of the subject based on the analysis,
wherein, while the optical sub-system is mounted on the wearable structure, the predetermined location is adjustable.

2. The system of claim 1, further comprising:
at least one actuator; and
a controller,
wherein the at least one actuator is configured, responsive to the controller, for adjusting the predetermined location.

3. The system of claim 2, wherein the optical sub-system comprises at least one light source being configured for the emitting of the light and at least one optical sensing element being configured for the sensing of the light, and
wherein the at least one actuator is configured for adjusting a position of the at least one light source and/or the at least one optical sensing element in relation to the wearable structure to thereby adjust the predetermined location.

4. The system of claim 3, wherein the optical sub-system comprises a plurality of optical sensing elements, the at least one actuator configured for adjusting the positions of the plurality of optical sensing elements independently from each other.

5. The system of claim 3, wherein the wearable structure comprises a platform and a static structure, the at least one optical sensing element being mounted on the platform,
wherein the at least one actuator is configured, responsive to the controller, to move the platform in respective to the static structure.

6. The system of claim 5, wherein the at least one light source is mounted on the platform.

7. The system of claim 5, wherein the radio transceiver sub-system is mounted on the platform.

8. The system of claim 1, wherein the optical sub-system comprises a plurality of light sources, each configured for emitting light in a respective direction,
wherein the predetermined location is adjusted by selecting a respective one of the plurality of light sources.

9. The system of claim 1, wherein the optical sub-system comprises a plurality of light sensors, each of the plurality of light sensors configured for sensing light from a respective direction,
wherein the predetermined location is adjusted by selecting a respective one of the plurality of light sensors.

10. The system of claim 1, wherein the radio transceiver sub-system comprises a plurality of antennas, each configured for emitting sub-optical radiation in a respective direction,
wherein the predetermined location is adjusted by selecting a respective one of the plurality of antennas.

11. The system of claim 1, further comprising a controller, the optical sub-system and the radio transceiver sub-system operated by the controller,
wherein, based on the analysis of the signals of the optical sub-system, the data processor is configured to generate an initial detection notification, and
wherein the controller is configured to control the radio transceiver sub-system to emit the sub-optical radiation responsive to the generated initial detection notification.

12. The system of claim 1, further comprising a controller, the optical sub-system and the radio transceiver sub-system operated by the controller,
wherein, based on the analysis of the signals of the radio transceiver sub-system, the data processor is configured to generate an initial detection notification, and
wherein the controller is configured to control the optical sub-system to emit the light responsive to the generated initial detection notification.

13-14. (canceled)

15. The system of claim 1, further comprising one or more additional sensors selected from the group consisting of one or more ultrasound transceivers, one or more electrical impedance sensors, one or more electroencephalogram (EEG) sensors, one or more electromyography (EMG) sensors and one or more temperature sensors,
wherein the data processor is configured to receive from the one or more additional sensors information regarding a respective attribute of the subject.

16. (canceled)

17. The system of claim 1, wherein the sub-optical radiation comprises a plurality of frequencies, a first of the plurality of frequencies being at least 10 times a second of the plurality of frequencies.

18-19. (canceled)

20. The system of claim 1, wherein the data processor is configured for delineating a boundary within the skull-contained matter at least partially encompassing a region having functional and/or structural properties that are different from regions outside the boundary.

21. (canceled)

22. The system of claim 1, wherein the data processor is configured to provide functional and/or structural information for each of a plurality of predetermined locations.

23. (canceled)

24. The system of claim 1, wherein the data processor is configured to analyze the signals to determine displacements of at least one of the optical and the radio transceiver sub-systems.

25. The system of claim 1, wherein the data processor is configured to analyze signals received from the optical sub-system to determine proximity between the wearable structure and the head.

26. The system of claim 1, wherein the data processor is configured to analyze signals received from the radio transceiver sub-system to determine whether the wearable structure is mounted on a living head.

27. The system of claim 26, wherein the data processor is configured to analyze signals received from the radio transceiver sub-system to determine dielectric properties of the tissue and to transmit control signals to the optical sub-system based on the determined dielectric properties.

28. (canceled)
